(19) United States
(12) Patent Application Publication
(10) Pub. No.: US 2016/0262717 A1
(43) Pub. Date: Sep. 15, 2016

(54) STETHOSCOPE AND ELECTRONIC DEVICE STRUCTURE

(71) Applicant: Clive Leonard SMITH, Englewood, CO (US)

(72) Inventor: Clive Leonard Smith, Englewood, CO (US)

(21) Appl. No.: 15/031,081

(22) PCT Filed: Oct. 28, 2014
(86) PCT No.: PCT/US2014/062594

§ 371 Date: Apr. 21, 2016

Related U.S. Application Data
(60) Provisional application No. 61/896,520, filed on Oct. 28, 2013.

(51) Int. Cl. A61B 7/04; H04R 31/00; H04R 1/46; H04R 1/10; A61B 5/00; H01R 24/58

(52) U.S. Cl. CPC A61B 7/04; A61B 5/0022; H01R 24/58; H04R 1/46; H04R 1/1025; H04R 31/006

(57) ABSTRACT

The present invention concerns novel extensions to the electronic stethoscope for physical construction, hardware design, software and signal processing, user interface design, and communications capabilities. In addition, a method for subscription services and methods is disclosed, which can be used for stethoscopes, medical instruments or any general electrical or electronic device or software application.

### FIELD OF THE INVENTION

The present invention concerns novel extensions to the electronic stethoscope for physical construction, hardware design, software and signal processing, user interface design, and communications capabilities. In addition, a method for subscription services and methods is disclosed, which can be used for stethoscopes, medical instruments or any general electrical or electronic device or software application.

### BRIEF DESCRIPTION OF THE DRAWINGS

FIG. 1 shows existing embodiments of electronic stethoscopes.

FIG. 2 shows one embodiment of the present invention.

FIG. 3 shows the internal structure of a preferred embodiment.

FIG. 4 shows an exploded view of the component stack inside the housing.

FIG. 5 shows the electronic stethoscope with expanded sensing.

FIG. 6 shows the functionality which may be associated with the connection jack on the stethoscope.

FIG. 7 shows the electrical connections that may be used on the connection Jack.

FIG. 8 shows the mechanical aspects of the jack connection.

FIG. 9 shows the power control on the connection Jack of the present invention.

FIG. 10 shows a table of the various functions that can be performed by a 4-conductor 3.5 mm or 2.5 mm standard audio jack and plug.

FIG. 11 shows the filter and signal processing settings available to the user, and controlled by the user, and the user interface design for such settings.

FIG. 12 shows a further user interface and method for setting or programming the signal processing capability of the stethoscope.

FIG. 13 shows digital signal processing, wherein the amplitude characteristics of different filters can be adjusted according to the frequency band of the given filter.

FIG. 14 shows attaching the stethoscope to a stethoscope style headphone or to regular headphones.

FIG. 15 shows a wireless link arrangements whereby the stethoscope can wirelessly communicate with wireless headphones or earphones and/or communicate with a mobile device such as a cell phone/tablet or computer.

FIG. 16 shows an implementation of a physical structure for the stethoscope wherein distinct modules can be stacked and attached to form a modular stack.

FIG. 17 shows another embodiment of the expansion capability, wherein an external module is plugged into the stethoscope.

FIG. 18 shows a further elaboration of the invention of an outside protective jacket for the stethoscope.

FIG. 19 shows a variety of user interaction methods envisioned by the present invention.

FIG. 20 further elaborates on data communications between the present invention and other equipment using audio signals as a means of data communication.

FIG. 21 shows a schematic form of encrypting and decrypting between digital form an analog form.

FIG. 22 shows another aspect of the present invention, which is to provide subscription devices that are enabled or disabled from performing certain functions based on a subscription being active or inactive.

FIG. 23 shows an alternative mechanical design for a stethoscope.

FIG. 24 shows one novel invention for use with the shaft stethoscope structure.

FIG. 25 shows the internal cavity in the shaft can be used to house functions beyond merely a battery.

FIG. 26 shows further novel expansion to a basic stethoscope structure.

FIG. 27 shows coupling means.

FIG. 28 shows variations on coupling means.

FIG. 29 shows the shaft fixed to the stethoscope base and the accessory attachment is inserted into the volume of the internal cavity of the shaft.

FIG. 30 shows a further mechanical structure for building a stethoscope.

FIG. 31 shows a neonatal stethoscope diaphragm comprising a diaphragm with protrusion.

### DETAILED DESCRIPTION OF THE INVENTION

In order to understand many of the novel improvements in the present invention, it is instructive to consider the conventional electronic stethoscope, as shown in FIG. 1, as a reference point for the prior art. FIG. 1 shows existing embodiments of electronic stethoscopes. In the existing embodiments, a chest piece, 1, is attached to a tubing, 2, which is then attached to a headset, 3. These parts form a complete contiguous and inseparable unit. In the existing embodiments shown in FIG. 1B, the electronic reproduction occurs through loudspeaker, 5, which transduces the electronic signal into a mechanical acoustic pressure which is then conducted through air up the hollow tubing, 7, to the ear tips, 8. The listener is therefore listening via the hollow tube, 7, to loudspeaker, 5. The disadvantage of this arrangement is sound quality is severely compromised by the transmission of acoustic signal mechanically via pressure in the hollow tubing, 7. The stethoscope is FIG. 1B forms a contiguous device and is separable below loudspeaker 5 only in order to occasionally change a battery located inside the stethoscope chestpiece structure.

The existing embodiment shown in FIG. 1A, is a significant improvement on the embodiment in FIG. 1B, in that the electronic audio signal is transmitted via a wires, 6, which are encased inside tubing, 2. The electrical signal is then connected to loudspeakers, 4, near the ear tips, 8. The quality of the sound is improved, by the fact that the loudspeakers, 4, are very close to the listeners ears and therefore there is very little acoustic loss of signal since the conduction of the audio signal is almost exclusively conducted via wires, 6.

The problem with both of the embodiments shown in FIG. 1, is that these conventional stethoscope designs suffer from being extremely cumbersome and very inconvenient for the user to carry or wear. In addition, the headphone structure is a custom headset designed to be part of a single contiguous device.

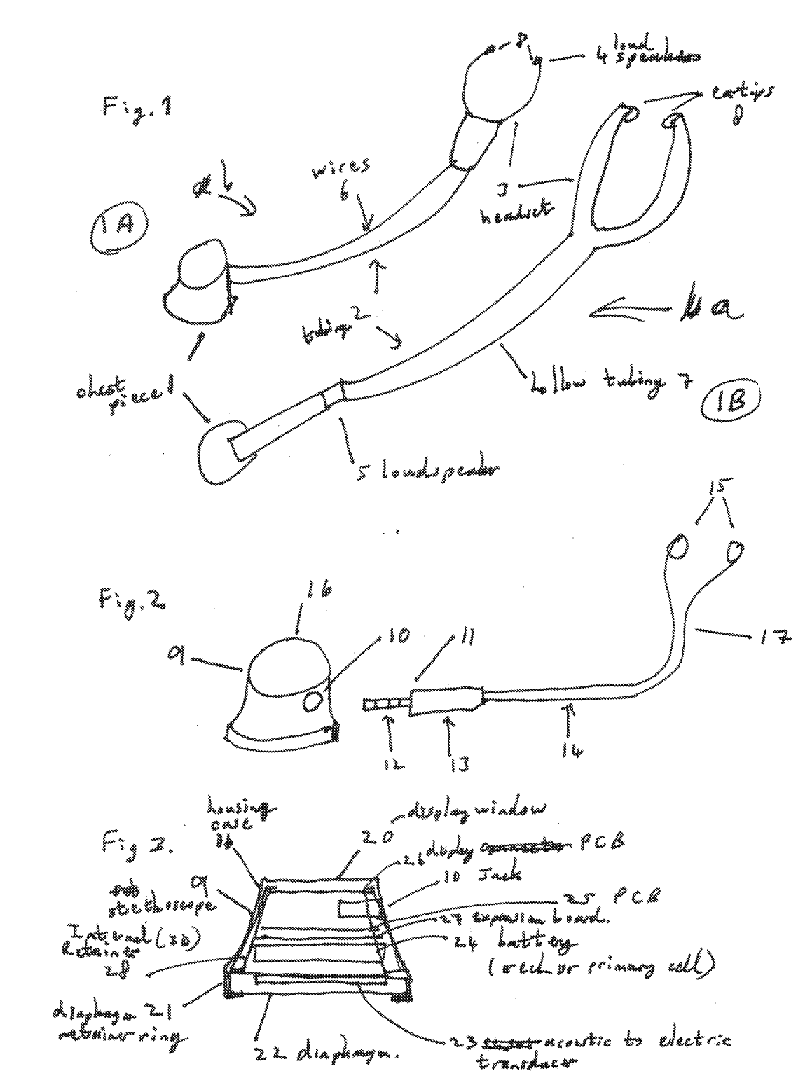

The present invention addresses this problem by creating a new embodiments, one of which is shown in FIG. 2. This shows one preferred embodiments of the present invention. In the present invention, the electronic stethoscope, 9, is completely housed within a single housing in a very compact form, being housed in a single case, 16. The electronic stethoscope, 9, has a connector, 10, which allows external headphones and other peripheral devices to be connected to the stethoscope, thereby providing substantial expansions to the functionality and features of the stethoscope while keeping the stethoscope extremely compact.

In order to listen to sound, external earphones or headphones, 17, can be plugged into the stethoscope, 9, via the connector, 10. The headphone plug, 11, has multiple electrical conductor connections, 12, which facilitates expanded functionality. The headphone plug housing, 13, provides the ability to facilitate a multitude of mechanical couplings between the headphones and the stethoscope. These mechanical couplings can provide a very convenience and quick method for connecting the headphones or detaching the headphones, or the mechanical coupling can provide a robust connection, which is resistant to inadvertent removal of plug 11 from stethoscope 9. The audio signal in this configuration is transmitted via electrical wires, 14, to headphones, 15. This provides the capability to transmit the audio signal electrically right up to the listener's ears.

The very compact electronic stethoscope, 9, shown in FIG. 2 represents a significant improvement in the mechanical construction of an electronic stethoscope and is a significant departure from the large bulky configuration that has been traditional in medicine. Such a small embodiment is a novel improvement to the conventional mechanical structure of the stethoscope. In the preferred embodiments, the connector jacks, 10, is a 3.5 mm, 4-conductor connection that is conventionally used in many audio devices such as cell phones and music players. Correspondingly, the headphone plug, 11, is a conventional 3.5 mm phono plug. One advantage of this arrangement is that headphones and earphones and headsets designed for general audio music listening and in common use with most cell phones can be conveniently connected to the electronic stethoscope, 9. This is a significant departure from the prior art as exemplified in FIG. 1, since in the prior art the stethoscope is a single unit and the headset is a custom attachment or part of the stethoscope. Even if the headsets can be decoupled from the chest piece it has not previously been possible to use any general-purpose headphones or earphones with prior art stethoscopes.

The dimensions of stethoscope 9 are groundbreaking in terms of their small size, with the electronics and all components fitting inside a space bounded by a cube that is less than 47 mm (Width)×47 mm (Depth)×29 mm (Height) or even 50×50×30 mm. It has not previously been possible to include an acoustic to electrical transducer, rechargeable battery, analog and/or digital signal processing circuitry, display, control and external connection means including expanded functions such as providing power to external peripherals, all in such a small package that fits into such a small volume.

FIG. 3 shows the internal structure of a preferred embodiment. The components and subassemblies of the stethoscope are mounted inside the external shell housing in a very compact manner. The elements are stacked inside the structure. At the bottom of the “stack” is the diaphragm, 22, which is held in place by the diaphragm retainer ring, 21. Above the diaphragm is the acoustic to electric transducer, 23. The transducer senses diaphragm motion and converts it to an electrical signal. Without loss of generality, the invention is not limited to the specific acoustic transducer shown in FIG. 3, (which is a capacitive acoustic to electric transducer), and the transducer function may be performed by various mechanisms including a microphone mounted in the lower portion of the stethoscope housing, or a transducer which operates on a principle that does not require the diaphragm as shown. For example, a piezo-electric transducer could be placed in the bottom section of the electronic stethoscope. All that is required is that a transducer be placed such that contact may be made with a patient's skin for the purposes of detecting body sounds.

Above the transducer are the other subassemblies and components required for the functioning of the stethoscope. A battery, 24, is located inside the housing. This battery is ideally a coin cell or other compact battery form factor. The battery technology can be either a primary cell or a rechargeable battery. Above the battery are circuit boards with circuits to perform appropriate stethoscope and other required functions. Above the battery, in the preferred embodiments, is a main printed circuit board, 25, which performs the main control and processing functions for the stethoscope. Functions can include analog and/or digital signal processing. Above this circuit board is the display circuit board or graphic display, 26. About the display is a display window, 20, which is made of a transparent material such as acrylic, glass, or sapphire crystal. The invention also allows for sufficient space inside the housing to allow for more circuit boards and more expanded functions to be included in the stack. Shown in FIG. 3, is an expansion board, 27. Such an expansion board can include functions such as wireless communications (such as Bluetooth, Wifi or Zigbee), wired communication protocols such as USB interface, physiological measurement functions such as ECG, blood oximetry, blood glucose, or provide other sensing functions. Such functions could also be included in the main printed circuit board, 25. The internal stack of components is preferably held in place by an internal retainer, 28. Such a retainer is preferably produced from plastic and may be produced by a 3-D printer, injection molding, or other plastic production process.

In another embodiment of the present invention, the outer housing case, 16, may be made from a low-cost material such as a plastic to form a disposable outer shell for the stethoscope. In this case the internal retainer, 28, houses all the inner workings of the stethoscope as if inside capsule, which is then covered with an outer shell when used clinically. The benefit of this structure is that the outer elements of the invention may be sterilized or provided as one time use disposable items, while the more costly inner workings of the stethoscope can be recycled and reused.

FIG. 4 shows an exploded view of the component stack inside the housing, 9. The same elements as were described previously in FIG. 3 are shown in this exploded assembly view. It is evident from FIG. 4 that assembly of the stethoscope in this embodiment is facilitated by this stacking approach, making it possible to very rapidly assemble the circuit boards into the housing along with the transducer, battery and other components. The entire assembly process using the stacked architecture can be reduced to a matter of a few minutes and also makes it possible to assemble the stethoscope using robotic techniques wherein a robot arm can pick each component and place it into the stack while simultaneously interconnecting circuit boards and other components.

It should be noted, that the same stacking architecture can be achieved by splitting the internal retainer, 28, into 2 mating left and right possibly mirror-image parts. The stack can then be inserted horizontally into one half of the internal retainer, 28, say the left side and then the enclosed by making the other half of internal retainer, 28, say the right side to form a closed retainer. The benefits of the stack are still maintained in this arrangement and such an arrangement can also be robotically assembled. Both approaches offer the benefits of a compact design wherein space is used extremely efficiently allowing sophisticated functions to be contained in a very compact housing, thereby making it possible to build a handheld stethoscope with completely self-contained functionality, electronics, acoustic sensing and power source.

The stacked design provides a further benefit in that vertically stacked circuit boards can be conveniently interconnected using board to board connectors.

The internal retainer, 28, further offers a number of novel advantages. It can be manufactured by 3D printing. This allows the internal retaining structure to be easily modified for different versions of the stethoscope. The inner design therefore becomes entirely driven by “soft tooling”, in that the mechanical structure can be modified on a computer and immediately printed on a 3D printer. The circuit boards can be modified, or software modified, to accommodate different functions and features in the stethoscope. Therefore a “family” of products can be manufactured without the need for injection molded tooling and the cost of changing a design becomes significantly lower than previously possible.

It is also to be noted that the rotationally symmetric outer housing, 9, lends itself to being economically manufactured on a high speed lathe when it is desired to manufacture the outer housing shell from a metal material. It is to be noted that's when the outer housing is manufactured using a plastic material injection molding, 3-D printing, or other plastic production process may be employed in which case the rotational symmetry is not necessary since plastic production techniques lend themselves to arbitrary shapes.

further aspect of the present invention is an electronic stethoscope with no internal source of electrical power such as a battery. A stethoscope which provides body sound sensing functionality, but does not contain its own internal source of power, may be powered from an external source such as a mobile phone or a computer. For example, a USB connection could be used to provide external power to the stethoscope during use. Such a stethoscope with no internal power source, which relies on external power and an interconnection, has extensive use potential in telemedical applications wherein the stethoscope is used to capture body sounds into an external mobile device to be transmitted to a remote listener. Another use case for and electronic stethoscope that is required to be externally powered, is when the stethoscope is to be used in conjunction with a separate mobile device. In such a case the stethoscope sensor is not intended to be used as an autonomous stethoscope in the conventional sense, but is intended to be used only as an external audio sensor for a primary device such as a mobile phone or tablet or computer, which manages most of the functions, such as filtering, audio amplification for headphones, and general control functions.

FIG. 5 shows the electronic stethoscope with expanded sensing. The sensing also includes user interface inputs, motion sensing, physiological sensing and optical functionality such as a light source and image detection sensors. For user inputs, pushbutton keys maybe placed around the sides of the housing, or touch sensors may be placed similarly on the side of the stethoscope or on the top of the stethoscope to provide touchscreen functionality. Also shown in FIG. 5 is an accelerometer mounted inside the stethoscope which provides motion sensing which may be used for user interface inputs, orientation sensing, positional sensing of the stethoscope on the patient's body or used in such applications as therapy such as cardiopulmonary resuscitation, in which case the accelerometer can be used to measure the rise and fall of the patient's chest. Physiological sensing functions may be included in the invention, including ECG electrodes placed coplanar or almost coplanar with the stethoscope diaphragm and placed either on the diaphragm or on the peripheral area such as on the diaphragm retainer ring. Image sensors can also be placed around the periphery of the diaphragm for sensing such physiological parameters as blood oxygen level or blood sugar level. An LED light source may also be placed around the periphery of the diaphragm or maybe placed on any other position on the stethoscope to provide a high-intensity light source they can be used for examining patients. The LEDs on a display, located on the top face of the stethoscope, could also be used as a general purpose light source, thereby combining the purpose of display LEDs to be both for informational display, and as a light source for diagnostic purposes. A camera may be placed on the stethoscope which may also be used for photography for dermatology or other images, capturing images for electronic medical records, or the camera could be used for pulse oximetry or blood sugar measurements.

Another element shown in FIG. 5 is an internal microphone inside the stethoscope housing. This microphone can be used for voice and ambient sound capture for recording or medical records and it can also be used to sense ambient sounds for ambient noise canceling functions, by using both the stethoscope sensor and the microphone as two sources of audio to be used for noise cancellation.

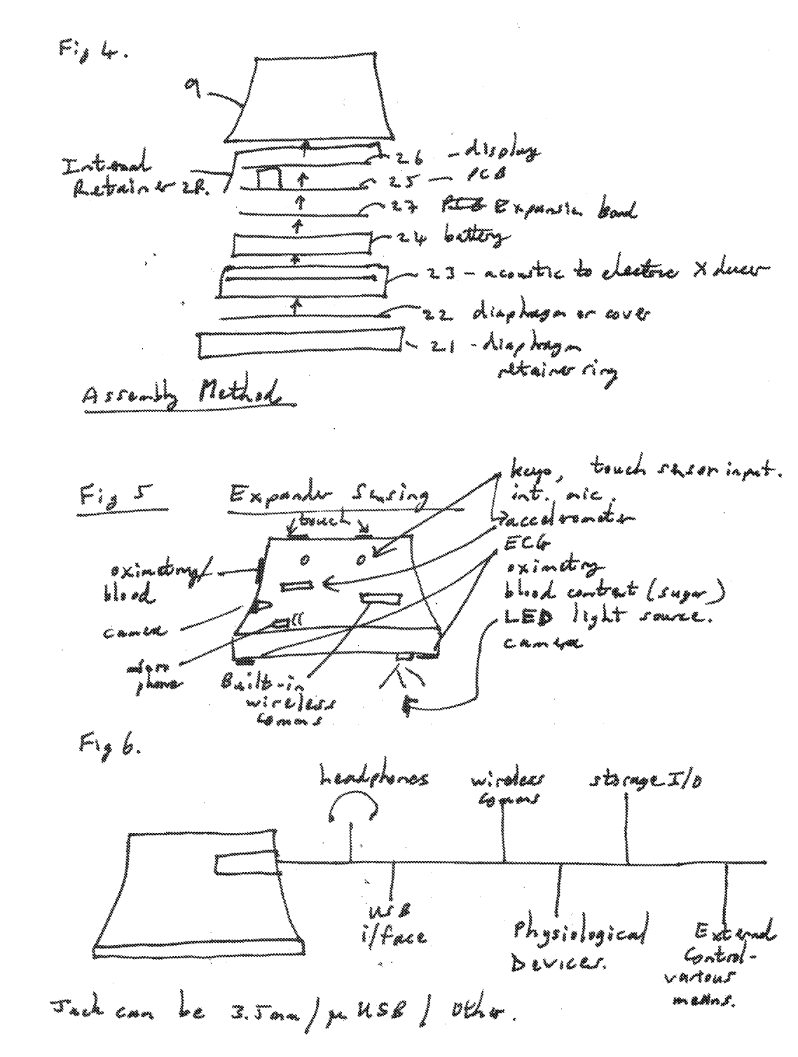

FIG. 6 shows the functionality which may be associated with the connection jack on the stethoscope. It is possible for the same Jack, with its multiple connections, to be used for headphones, a wireless communications device, external storage for writing data to the stethoscope or reading and storing data from the stethoscope, a USB interface device, external physiological measurement devices including those that have been listed previously, or a connection for external interface and/or control by various means including cell phones, computers, or tablets. It is also possible to connect external devices, which would provide interface capability between the stethoscope end and mobile devices including cellular telephones, tablets, and computers. The connection Jack is therefore a multipurpose interface providing extensive expansion capability to the stethoscope while maintaining the compact physical form factor of the self-contained handheld stethoscope. In the preferred embodiments, the connector is a 3.5 mm phono connection which is commonly used on other mobile devices such as cell phones. However, the same functionality and convenience can be achieved by using other common connector form factors such as micro-USB or other connections.

FIG. 7 shows the electrical connections that may be used on the connection Jack. As shown in FIG. 7, by example, they offer connections on a 3.5 mm connection. There is a left audio channel, a right audio channel, a ground connection and a power connection. The ground connection is intended to provide the electrical ground reference for all other signals. The left and right connections are used for audio output from the stethoscope to external devices or headphones or for audio input into the stethoscope. The power connection is intended for power inputs to power the stethoscope or to charge the internal battery. Power outputs can also be achieved to allow the stethoscope to power the external peripherals which are connected to the Jack. These peripherals were described previously and include communications devices, physiological measurement devices, digital storage devices and other items listed previously. The signals on the connection Jack can also be multiplexed to perform multiple functions. For example, while the left channel connection could be used for audio output of stethoscope sounds, the right channel connection can be used for bidirectional serial communication using either analog signaling or digital signaling. In such a configuration, an external device can control, program, and configure the stethoscope, either in a special operating mode or while the stethoscope is been used to listen to patients. Other functions may be multiplexed onto these pins/connections such as Digital audio serial streams, USB, or other high-speed digital communications. The purpose of such a connection is to provide extensive expansion functions, while at the same time using the same jack for a simple headphone connection.

FIG. 7B shows that the same functionality or even more extensive functionality may be achieved using a different connector such as a micro USB connection. Such a connection can provide analog audio, digital audio, digital communications, power input and output, and remote control. The present invention, while illustrating a 3.5 mm jack and a micro USB jack, may also be implemented using numerous other interconnections, wherein 4 or more conductors are available for connection to the outside world for audio, digital and power transmission.

FIG. 8 shows the mechanical aspects of the jack connection. The Jack not only provides electrical connection between the stethoscope and external devices, headphones and cables, but also serves to secure the connection mechanically in a robust manner to ensure that peripherals do not fall off or that connections are not broken when the stethoscope is used in rugged situations with pulling, tugging, shaking, etc.

FIG. 8A shows a screw thread on the connection in which case the male plug would have a thread to secure the connection mechanically. FIG. 8B shows a friction fit wherein the plug is inserted and held in place by frictional forces. FIG. 8C shows a bayonet arrangement, in which case the male plug has protrusions which enter into the slots, and the plug is then rotated to secure the bayonet connection. In a somewhat similar manner FIG. 8D shows clips attached to the male plug, which would be spring mounted and engage the opening and lock the plug into place.

These mechanical arrangements are especially suited to a stethoscope wherein it is desirable for a more conventional stethoscope-style headphone to be attached to the stethoscope. In this case the stethoscope is electrically and mechanically attached to an external stethoscope-style headphone, which may have a cable or tubing which is then connected to a more conventional stethoscope-style headphone with stiff arms for the left and right headphone. In such cases, a user might wear a stethoscope around the neck, and emergency medical worker might be in a hostile environment and this mechanical arrangements insurers that the small stethoscope described in this invention and shown in FIG. 2, item 9, would not get lost or fall off the stethoscope style headphones.

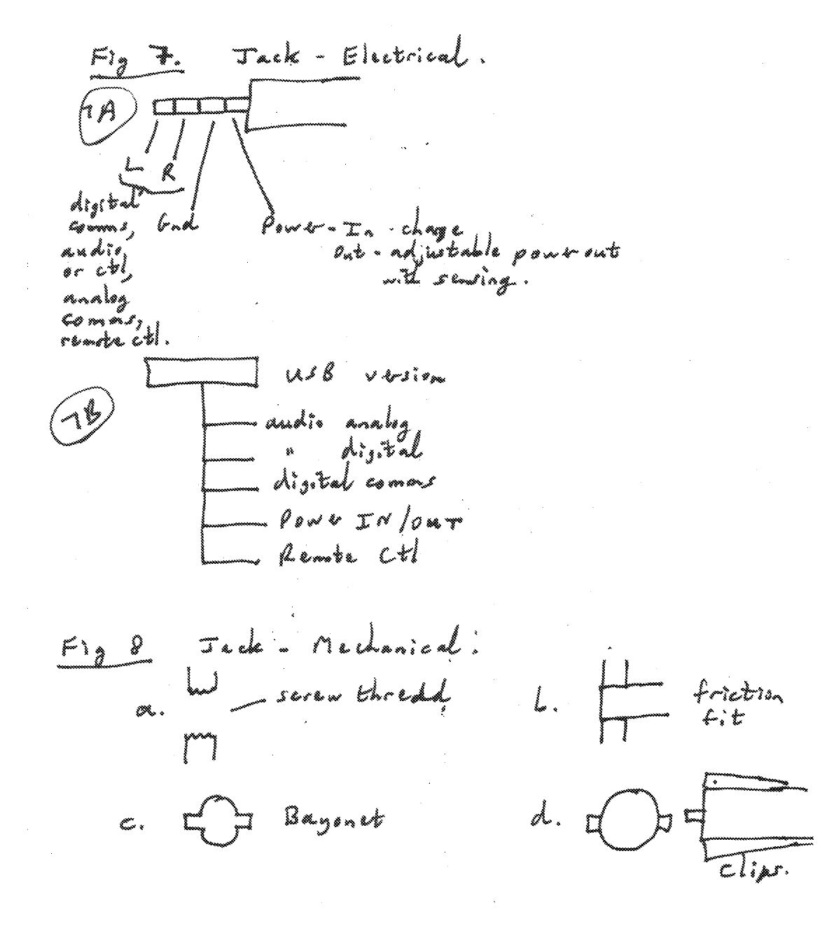

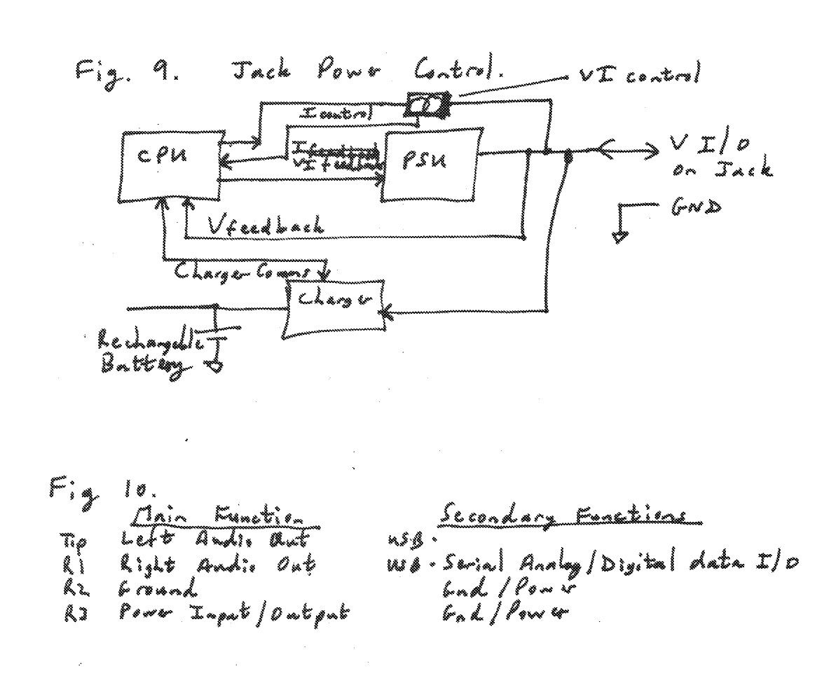

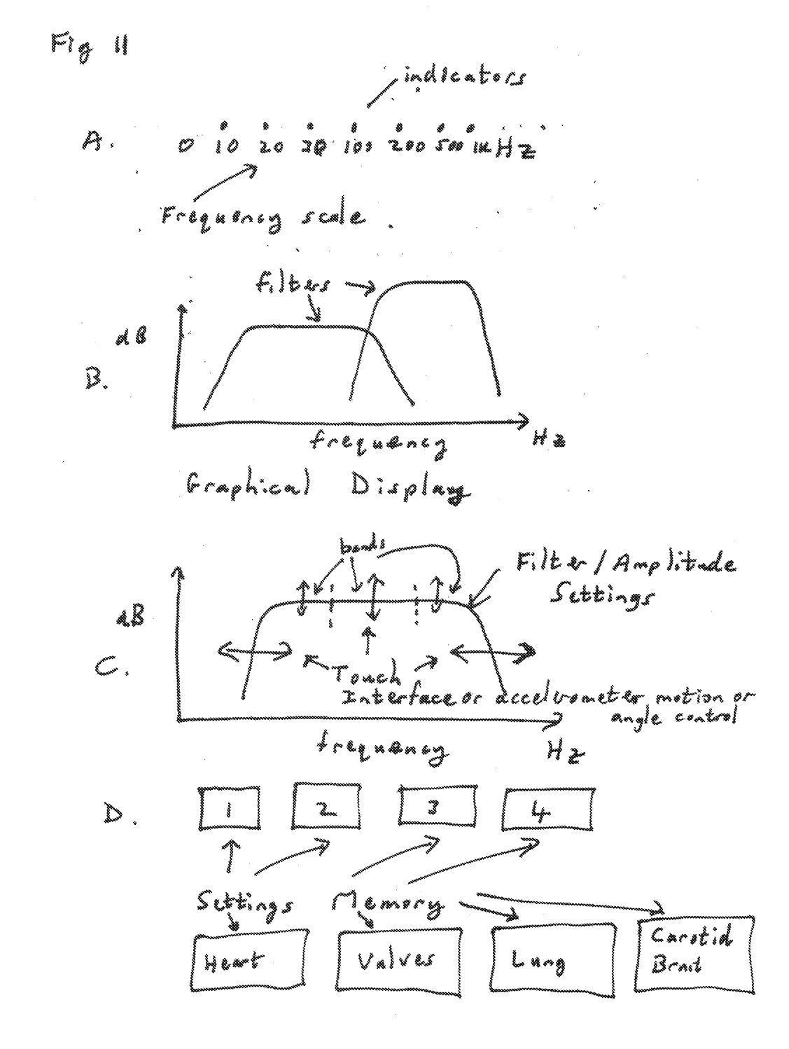

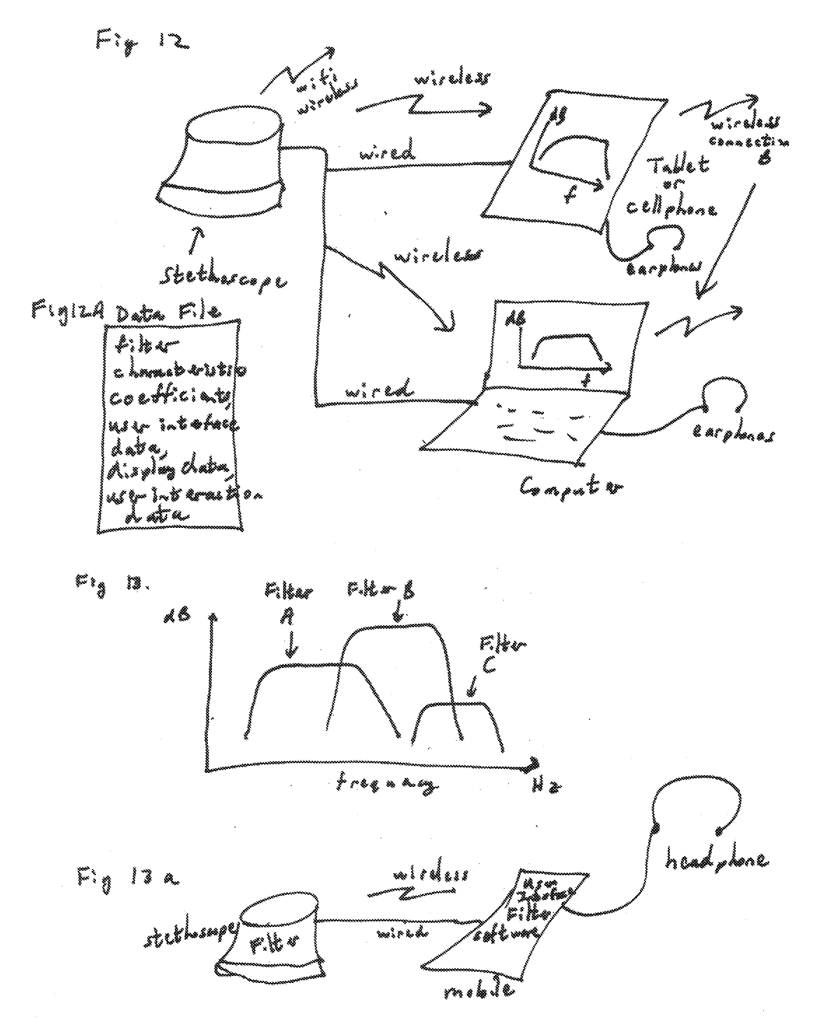

FIG. 9 shows the power control on the connection Jack of the present invention. There is a power terminal and a ground terminal connected to the jack connection. The power connection can either supply power to an external device or power can be input into the device from an external power source, depending on the particular mode of operation. When power is supplied from an external power source, it is typically used to either power the entire device and charge the battery, or merely to charge the battery. There is a charging circuit, which controls the charging of the battery from the external power source, typically regulating the voltage and current levels to the rechargeable battery, subject to the requirements of the particular battery chemistry. The charger circuit is also controlled typically by a central processing units or micro processor. The microprocessor can monitor the status of the charging process and it can enable or disable the charging process.

When the power connection on the Jack is used to supply power to an external device it is necessary to control the delivery of power to the external device. The present invention includes current and voltage control to the external device allowing the central processing units to determine and decide what levels of power are appropriate for delivery to an external device. In one embodiment, the simplest embodiment, a power supply is typically a single semiconductor device such as a single output voltage regulator chip, supplies a regulated voltage to the power connection. This power may be delivered on a “dumb” basis or the central processor unit can enable or disable the power supply automatically or as a result of manual control by the user or data communications with an external device. In a more sophisticated arrangements, a voltage or current control circuit is controlled and monitored by the central processing units through voltage or current feedback to the central processing unit. A limited current can then be supplied to an external device, for low-power devices or devices in which a limited current is necessary, or the output resistance or output impedance of the voltage or current supply can be adjusted in order to regulate the voltage or current being delivered an external device. In one embodiment, voltage through a resistor connected to the external power connection can be switched on or off and the resistance changed between a voltage supply and the external device. By being able to measure the voltage and current characteristics of the external device connected to the power connection, a central processing units can then determine the amount of power required by the external device and thereby determine whether to enable the high current power supply units or disable it and provide a throttled or limited level of power to an external vice. In summary, the delivery of power to an external device can be controlled by controlling the output resistance, voltage or current of the power output connection. The circuit can optionally measure the current, voltage and impedance characteristics of the load presented by the external device, thereby determining what power output characteristics to present to the external device.

further advantage of the voltage and current circuit arrangement with feedback to the central processing unit is that the central processing unit can use the voltage and current characteristics of the external device as a form of identification. For example, different values of resistance could be presented by an external device to the power output line on the jack, and by determining the amount of current at a given voltage that is flowing into the external device from the present invention, the central processing unit can then use an internal algorithm or a lookup table to identify a general category or class of peripheral device presently connected, or it may make a very specific determination as to the nature, category or specific identification of the peripheral device. Alternatively, a low power, low current output can be enabled that supplies just enough power for the external device to provide a digital signal input to the device to identify itself, at which point the power control algorithm can determine which power setting to apply to power the external device.

The bidirectional power supply capability illustrated in FIG. 9 allows the present invention to be charged from an external power source, and to provide power in a regulated manner to external devices and equipment. Such external devices may include wireless communication attachments, external headset to audio control attachments, interfaces to mobile devices such as cell phones and tablets and laptop computers, external physiological measurement devices as listed previously, external digital devices for storage and data communications.

The voltage current characteristics of an external device can also be used as an enabling or disabling function, in which specific voltage and current combinations or load impedance characteristics of the external device can be used as a form of identity in order to categorize peripherals as either being permissible for use with the present invention or locked out and blocked from use. Device designers can, by this mechanism, set specific resistor values which the central processing unit measures and identifies and uses those specific values to determine whether to communicate with the external peripheral or perform a lockout or disabling function in order to prevent a given peripheral from being connected. With this simple mechanism the central processing unit can ensure that only approved devices may be used in combination with the present invention. Very specific resistance values could be used as unique identifiers for approved devices. Alternatively, a specific voltage or current level could be presented to the power connection on the Jack that specific current or voltage measured by the central processing unit thereby using voltage or current as a unique identifier and enabling or lockout mechanism. The unique resistor values that are used could be designed to be values that are not part of the standard 5% or 1% resistor values currently used in the industry, and could be resistances that are required to be constructed by series or parallel combination of standard resistances, in order to present a resistance value, current value, or voltage that is not typically used or available from off-the-shelf components. By using unique resistance, current or voltages as an identifier, such values could become “trademark” identifiers of acceptable external devices.

The above power control method is applicable to a broad class of devices beyond merely stethoscopes and can be applied to cellphones, tablets and many other smart devices or peripherals.

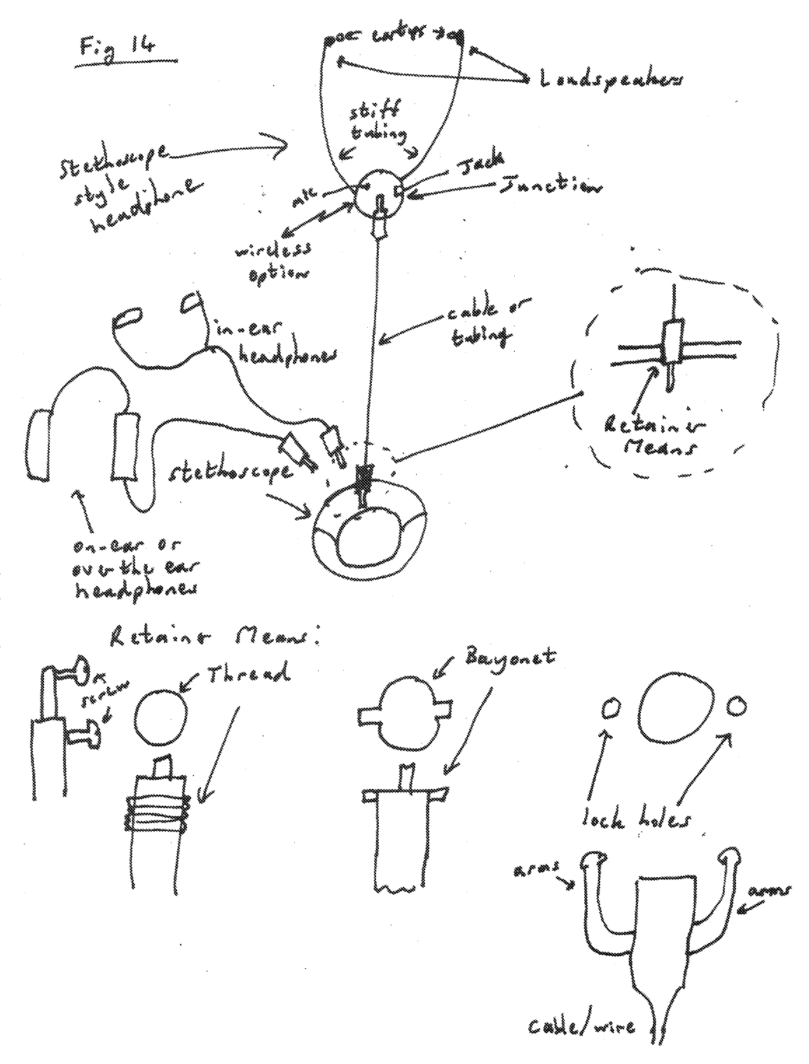

FIG. 10 shows a table of the various functions that can be performed by a 4-conductor 3.5 mm or 2.5 mm standard audio jack and plug. It should be noted, however, that other standard connectors may be used for the same purpose such as a USB, micro USB or other custom Jack and plug arrangement. In the case of using a 3.5 mm or 2.5mm 4-conductor connection, the main function connections can be left audio output, right audio output, ground, and power input/output as described about. The order in which these are listed are tip, ring 1, ring 2, ring 3, although other connections are possible. These connections may be switched or multiplexed to perform other functions. The left and right audio analog signals could be switched to become USB plus and minus signals, facilitating an all-digital communications link via a standard headphone jack. In such an arrangement, a user could plug in standard headphones and listen to analog audio, or could connect a USB interface to an external mobile device or computing device and perform digital audio and data communications. Another alternative is to provide analog audio outputs on one connection and use the 2nd audio channel instead for serial analog or digital data communications. Such data communications could be in one direct and only or both directions and could take the form of an analog data communications signaling method or a digital data communication signaling method.

It should be noted that while a single jack is preferred in the present invention, especially in order to achieve small size and reduce cost, the multitude of audio and digital data communications methods, interconnections to peripherals, charging methods, and delivery of power to external devices or from external devices, may be implemented using more than one connection. It should also be noted that communication of analog information and digital information, audio signals, and power signals, can all be implemented using wireless connections.

In the case of wireless power transmission, inductive, magnetic, or capacitive coupling can be performed between an external charger and a charging circuit connected to an internal rechargeable battery. Other over the air wireless recharging techniques are also envisioned by the present invention. In this case, the stethoscope could be placed on a charging pad, a docking device, or placed physically in proximity to a wireless signal which is coupled to the charging circuit inside the stethoscope. Similarly, audio and data communications signals could be transmitted to or from the stethoscope using standard digital data communications over wireless nature such is Bluetooth audio, Bluetooth data, Wi-Fi, or other custom or wireless standards.

FIG. 11 shows a novel aspect of the stethoscope invention, which are the filter and signal processing settings available to the user, and controlled by the user, and the user interface design for such settings. The present invention utilizes analog and/or digital signal processing to implement audio filters suitable to the processing of heart, lung and other body sound signals. In the conventional stethoscope, mechanical or electronic, it has been the tradition to provide a limited number of filters, which cannot be adjusted by the user. There is usually a filter for low-frequency listening, typically named “Bell”, and a filter for higher frequency listening, typically named “Diaphragm.” The Bell filter is usually used for heart sounds and the Diaphragm filter is used for lung sounds or listening to heart valves. In some electronic stethoscopes, there is also a full frequency filter sitting, which allows a wider range of audio frequencies to be transmitted to the listener.

In FIG. 11, a more advanced and flexible filter and/or volume setting mechanism and user interface is presented. This is a significant departure from the conventional bell and diaphragm modes. In FIG. 11A, a frequency scale is shown, wherein the range of frequencies being passed by the signal processing circuitry and software is indicated by indicators, typically LEDs, on a horizontal frequency scale. Optionally, the scale can be identified with specific numeric measurements, typically measured in hertz, or the specific scale numerical indications may be omitted, and the indicators showing merely the range of frequencies in a simplified manner to the user, indicating that lower frequencies, higher frequencies, or other selected ranges of the frequency scale are being transmitted or filtered by the signal processing function. The user may set these filter settings and customize the frequency ranges to her own preference, and save the settings in memory. Alternatively, a variety of filters may be preset and offered to the user, providing an easy mechanism for the user to select which frequencies to use for listening to a given physiological phenomenon. The presentation of a frequency scale on a stethoscope, as shown, is novel. Previously, much simpler indications are provided for Bell, Diaphragm, and/or Full range.

In FIG. 11B, a more graphical display of the signal processing functions and filters is shown. In this more graphical display, a more accurate representation of the filter bandwidths and amplitudes can be indicated, with the bandwidth shown along a frequency scale and the amplitude shown along a magnitude or decibel scale. As the selected filters are changed, the particular filter being used can be shown or highlighted. Such filter characteristics are known as “transfer functions” or “Bode plots.” This display could show multiple transfer functions of the various filters that are available to be selected by the user, allowing the user to select a particular filter from more than one filter. Thus, the graphical user interface could be used to present multiple filters for selection by the user, or it could be used to indicate the particular filter being used at the present time. Alternatively, multiple filters could be shown, with the present filter that is being used, being highlighted.

FIG. 11C shows a further enhancement to the graphical user interface wherein the user can use a touchscreen to drag the transfer function plot to suit his or her particular listening needs. The lower frequency cutoff frequency can be dragged left or right, the higher cutoff frequency could be dragged left or right, and the volume or amplitude can be dragged up or down with the amplitude being uniform across the passband or the amplitude being divided into segments such that some bands are louder and other bands are quieter.

The dragging operations may be performed via a touchcreen, but alternatives are also included. The built-in accelerometer may be used, wherein the angle of the stethoscope can be used to shift the cutoff frequencies and/or amplitude curves. Another accelerometer method is to use shake actions left, right, up or down to “click” curves in a given direction.

FIG. 11D illustrates the ability to save any filter, volume, amplitude, or signal processing settings in specific memory locations for easy recall at a later time. The settings memories can be labeled numerically or they could be labeled in association with a particular clinical diagnostic function such as heart, valves, line, carotid bruit, or other particular labeling that is meaningful in a clinical setting, using text names for each setting memory.

The user interface described in FIG. 11 is displayed on a display that is placed on the stethoscope itself, on the top surface in the preferred embodiment. Alternatively, the user interface is done on a mobile device or computer and the setting transmitted to the stethoscope, or implemented in the mobile when the audio stream or signal is received, or received and retrieved from mobile memory.

FIG. 11 illustrates the advances in capability of digital stethoscopes and digital signal processing over that of the prior art currently in use. The bell and diaphragm paradigm reflected the very limited mechanical filtering capability of mechanical stethoscopes. Even when electronic stethoscopes came into use, the conventional wisdom was that the user interface must maintain the old paradigm and a simplified set of options must be presented to the user. The user interface of electronic stethoscopes has therefore never provided the user with the enhanced options made possible by digital signal processing. The present invention is novel in that it provides the user with a far more powerful set of options for setting the filters on a stethoscope and for customizing filters for both specific clinical applications and for the user's particular listening needs. Beyond the clinical categorization and classification of filters and signal processing, the user interface and filters setting capability can also be used to adjust the stethoscope signal processing to suit the hearing and audiological needs of the listener, adapted for the hearing deficits of a particular user.

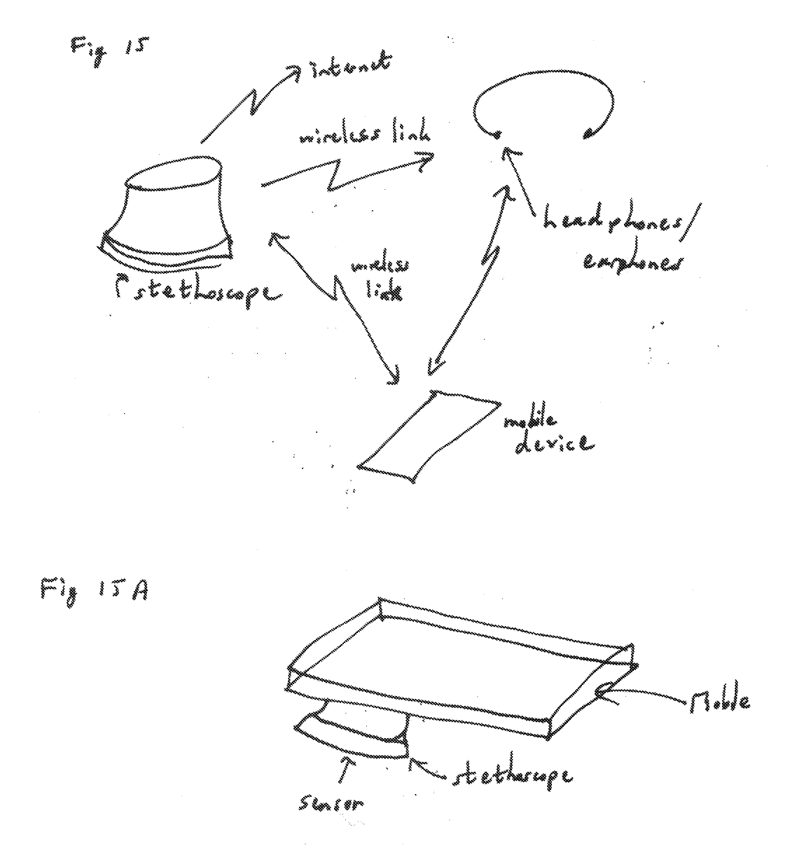

FIG. 12 shows a further user interface and method for setting or programming the signal processing capability of the stethoscope. In this arrangement, the stethoscope is connected via a wired or wireless communication link to a tablet a cell phone or a computer or via a Wi-Fi or wireless link to a remote computer system. In this arrangement a user may select or program desired stethoscope signal processing settings on a tablet, cell phone or computer, or access such settings over a wireless link directly to the stethoscope. Such filter settings could be based on available filters from a group or library, or customizable filters which can be selected or set on the user interface means, said means being the tablet, cell phone, computer, or remote server. The settings can be based on clinical needs or definitions such as heart sounds, lung sounds, carotid bruits, bowel sounds, fetal sounds, etc. More specifically heart sounds settings could be more distinctly classified under 3rd heart sound, heart failure, valve sounds, murmured detection, murmurs, etc. Lung sounds could be classified as a disease classification such as pneumonia or asthma, or could be classified as wheeze, crackles, rhonchi, etc. Alternatively, the user can adjust and customize the filter characteristics for specific requirements as discussed previously and shown in FIG. 11, the difference being that such a user interface means, such as menu selections, keyboard entries, or touch interface settings could be done via a tablet, cell phone or computer.

FIG. 12 further shows the potential to adapt and adjust the signal processing characteristics of the stethoscope to the specific requirements and audiological requirements of a hearing impaired user. In this case, the clinical or physiological classifications could be further adjusted by the hearing impairments of a specific user. FIG. 12 shows earphones connected to the tablet, cell phone, or computer (hereinafter also referred to as “mobile device”). A hearing test can then be implemented on said mobile device, and the results used to adjust the signal processing and digital filtering settings in the stethoscope accordingly. Specifically, the volume settings on the stethoscope could be adjusted so that the range of volume and amplification, as well as the specific frequency band amplitudes, can be adapted to accommodate any deficiencies in the user's hearing. Alternatively, the data from a hearing test done by a professional or done previously by the user, can be downloaded into the filter adjustments data in stethoscope programming software, or imported from an external source, said data being used to modify the stethoscope signal processing and filters according to a previously or separately performed audiology test. The method thus enables the user to adjust the filtering characteristics of the stethoscope filtering or signal processing to be customized according to a hearing test, administered either locally at the time of adjustment to the stethoscope by the user, or based on an audiology test administered previously by the user or an audiology professional.

The adaptation filter that is applied to customize the hearing preferences or deficits of the user can be applied as a “global transfer function” that is applied to standard filters. So the user can program the adaptation to his/her hearing needs, and separately select particular filters according to previously described selection preferences for heart, lung, etc. The same global adaptation can be done for any reason, including the listener's personal preferences.

FIG. 12 a shows a data file or data structure the contents of which serve to specify the digital filters or digital signal processing functions as they are downloaded or transferred into the stethoscope from an external device. It is customary for digital filters to be defined by coefficients or data tables. The present invention expands upon the data structures to include these coefficients as well is user interface data, display data, and user interaction data. In this case, it is intended that the term data means any information required by the stethoscope software. The data structure for a given filter therefore includes elements such as which LEDs would be lit in FIG. 11A in a representation of the frequency response of the filter, or how a given filter would be rendered graphically as shown in FIG. 11B or FIG. 11C and/or information such as the labels or names that would be used for a given filter setting. Such a comprehensive data structure thereby provides a rich data sets wherein the digital signal processing specification to be used by the stethoscope defines not only the signal processing characteristics, but also the colloquial terminology or labels that would be used by the user, or display settings that would facilitates a user-friendly stethoscope user interaction experience. A further elements of information that is included in the filter data file or digital signal processing specification also include the elements of the user interaction with a stethoscope. For example, the information might include which are the most preferred signal processing or filtering settings, what the sequence of switching would be between any given filters, and other data which facilitates the use of the stethoscope and the setting of filters. For example, the user may have a particular filter that is to be used by default or when the stethoscope is first turned on, or the user may have two filters which are primary favorites among a larger set of filters functions, and the data structure includes such information so that the user can easily switch between favorite filters and then with further actions, dig deeper into the folder library in order to use a less favorite filter.

To clarify, the data files or structures therefore include not only the signal processing coefficients or algorithm, but also user interface information as to how the filter selection will be presented on the display, as well as user interface behavior such as identification of default and favorite filters, the order in which filters are offered when scrolling through a selection, the labels or names of the filters, and other data that drives the user interaction with the stethoscope when operating and selecting filter and volume settings.

The present invention therefore provides a very novel capability for processing audio signals in a stethoscope, providing an expanded set of filters, and an expanded set of adjustments within each filter, and multiple means of selecting and setting those filters. The present invention also offers a simplified means of selecting such filters, either by providing a set or library of filters or signal processing functions saved into the memory of the stethoscope, or libraries and functions which may be downloaded from the Internet and/or transferred from mobile devices to the stethoscope.

FIG. 13 shows a further benefit of digital signal processing as implemented in the present invention, wherein the amplitude characteristics of different filters can be adjusted according to the frequency band of the given filter. Such adjustments may be made and preprogrammed or later programmed into standard filters or custom filters saved in the stethoscope memory and performed by the signal processing circuitry. It is well known that human hearing is not uniform with respect to frequency and sensitivity across the entire audio spectrum. These variations have been characterized by what are known as Fletcher Munson curves and these variations are also psycho-acoustic. Shown in FIG. 13, are 3 filters, for example purposes only, wherein both the bandwidth and the amplitude difference for each of filter A, filter B, and filter C. such settings take into account the possibility that different frequencies are emitted by the human body in different proportions. By allowing the amplitude settings to change between filters, a given volume setting on the stethoscope can be made to provide a uniform listening experience for the user. So in the illustration, it may be that lower frequency signals from the human body that would be passed by filter A are much greater in amplitude than mid frequencies that would be passed by filter B. By setting filter A amplitude to be lower then filter B amplitude, the listener would perceive the body sounds passed by filter A to be equal in loudness to those passed by filter B. Therefore, by allowing the filter amplitudes to compensate for both psycho-acoustic phenomena and the fact that different frequencies are emitted by the body at different levels of loudness, the user can listen to signals at different frequencies at a given volume setting level on the stethoscope, and find that volume setting to be comfortable for a range of differing filter settings. If this were not the case, for a given volume setting of the volume control, the sounds being passed by one filter may sound uncomfortably loud while sounds passed by a different filter would be too quiet.

FIG. 13 a shows a further arrangement for signal processing stethoscope sounds by using an intermediate mobile device such as a smart phone, tablet, or computer, all indicated by the label “mobile” in FIG. 13A. In this arrangement stethoscope sounds are optionally filtered or partially filtered inside the stethoscope and transmitted to the mobile device, either via wire or wirelessly, and further filtering software executes further signal processing within the mobile device, the result being reproduced via headphones for the listener. In this arrangement, the stethoscope user interface is largely performed via the mobile device, utilizing the power of user-friendly apps and user interactions that are possible with mobile devices and touchscreen interfaces. Therefore the stethoscope becomes a peripheral audio sensing device for the most part, and many of the user interaction functions and digital signal processing functions are performed in the mobile device. Optionally, such functions could be shared between the stethoscope and the mobile device. By offloading many of the features of the stethoscope into the mobile device, significant cost savings are possible in the actual hardware required for the stethoscope. The invention therefore includes the capability to split functions between the stethoscope and mobile device.

The departure of the filter selection process presented above from that of prior stethoscopes is a significant novel step. Historically, stethoscope user interfaces have maintained the Bell and Diaphragm paradigm for the purposes of simplifying the choices presented to a busy clinician. It has always been maintained by stethoscope designers, that the bell and diaphragm filtering paradigm is so ingrained in medical practice that a more complex presentation of filtering options, in which clinicians are required to understand concepts such as frequencies, bandwidths, and frequency response, completely contradict the need of the clinician to be able to work quickly, use a simple user interface, and be able to associate the functions of an electronic stethoscope with those of the mechanical stethoscopes previously used. It has commonly been taught that the digital filtering and signal processing in an electronic stethoscope should as closely as possible match, model or imitate the audio and frequency characteristics of traditional mechanical stethoscopes. The bandpass filters that have previously been used in electronic stethoscopes have therefore been labeled as bell or diaphragm and have been designed to match the very specific frequency response characteristics of particular bell and diaphragm characteristics of specific mechanical stethoscopes, or at the very least, an ideal version of a bell or diaphragm transfer function. The present invention departs from this prior teaching in that modeling the digital filters in an electronic stethoscope to match those of either ideal or actual mechanical stethoscopes suggests that the imperfections of mechanical stethoscopes that have been used for more than a century are actually desirable in newer implementations of the filtering function. However, conventional mechanical stethoscope filtering and audio transmission characteristics are severely hindered by the limitations of transmission of sound via air tubing to the listener's ears, as well as the limitations on the reasonable shape and dimensions of the bell and diaphragm of the mechanical device. Therefore, to create digital filters that match, model or imitate such imperfect transmission characteristics negates the benefit of allowing the creation and setting of filter parameters that are based on filter characteristics including lower cutoff frequency, upper cutoff frequency, bandwidth, amplitude settings, and the like. The inventive step is to dispense with prior filter characteristics and settings and base filters on bandwidth and transfer functions as more pure mathematical concepts, rather than models of mechanical stethoscopes.

Regarding the user interface, electronic stethoscopes, previously being limited to settings such as Bell, Diaphragm or their clinical equivalents Heart and Lung, impose an overly simple set of options. Therefore, the method disclosed in the present invention, in which various means are provided for showing filter bandwidth on a frequency or Hz scale or graphic display, is a novel user interface design never before presented on a stethoscope.

Another unique feature of the present invention, given the capability to create customized or specific signal processing methods or digital filters, is that the invention offers methods for sharing or distributing specific filters, set and designed for very specific applications, or to the preference of a given user. Therefore, another unique aspect of the present invention is a method for sharing or distributing digital filters. The steps are as follows:

user selects the specific characteristics of a filter, specifying it's parameters as permitted by one of the user interface methods previously described. Next, the user saves this setting, optionally providing a name for the filter. The name can be arbitrary, such as named for a given clinician, or named for a given pathology, etc. Such naming is up to the user. The next step is to save the filter and optionally to upload the filter to a server, or send the filter data via an electronic communications means to one or more other users. The recipient, or a visitor to a website can then download the filter so designed, and load it into his/her own stethoscope memory, using one of the communications methods previously described. This process therefore provides a method for defining a signal processing algorithm or digital filter, saving it, uploading or sharing it, and enabling others to load the algorithm or filter into other stethoscopes.

One application for such sharing of filters might be in the educational setting, in which an educator can define a filter which is very specific and enhances a particular body sound in a particular way, to facilitate better learning by a student. In another application, researchers may design a filter that meets the needs of a unique research program, and they wish to distribute the filter to other team members who need to make the same measurement. A third need for such a filter would be a company, such as a Left Ventricular Assist Device (LVAD) manufacturer that defines a filter that is specifically tuned to detect the sounds from the LVAD for the purposes of detecting failures or decreased performance of the LVAD. The manufacturer can then send the specific filter into the field for clinicians and engineers in the field to use for detecting specific LVAD performance. There are, therefore, many applications in which it is desirable to define filters and make them available to other users for download into their stethoscopes. In all these applications, the conventional approach wherein filters are limited to Bell or Diaphragm or other such simple category, entirely negates the potential to tune stethoscope sound using digital signal processing and digital filtering.

Yet another method for loading new filters into the stethoscope, is a method wherein a digital filter designer uses a third party software package, such as The Mathworks' Matlab software, to design the coefficients of a filter, or to design an entirely new algorithm for signal processing of stethoscope sounds. In developing an algorithm, computer code written in a high level or assembly language might be necessary or a tool such as Simulink can be used to automatically generate code. In either case, the steps in the method are to develop the coefficients for a digital filter, or the code for a complete algorithm, and then to download the code or coefficients into the stethoscope signal processing hardware or software memory in the stethoscope, to be stored and executed or used for processing body sounds. This is the most difficult means of developing new algorithms of filters, however the present invention provides for such capability in that the stethoscope includes a means for communicating digital information, data, code and software from an external device to the stethoscope memory. Note that such methods can include all the steps disclosed in this invention for digitally communicating information between the stethoscope and other devices such as mobile devices, computers, external memories, servers and other external devices and systems. The communications link is therefore used to transfer filter and signal processing algorithms and data into the stethoscope.

The present invention allows for different models or versions of the stethoscope to be programmed to offer different levels of filtering permissions. Therefore, the same stethoscope can be programmed to allow only one filter setting, whereas another model can be programmed to allow for multiple filters to be programmed. This allows for price differentiation via software and filter permissions.

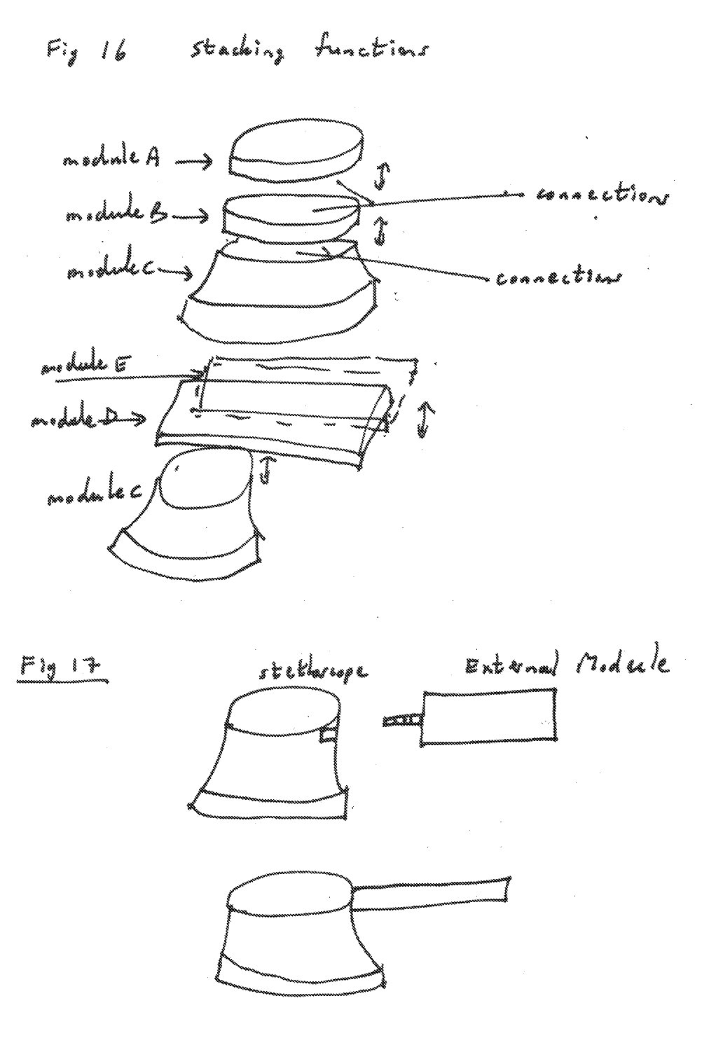

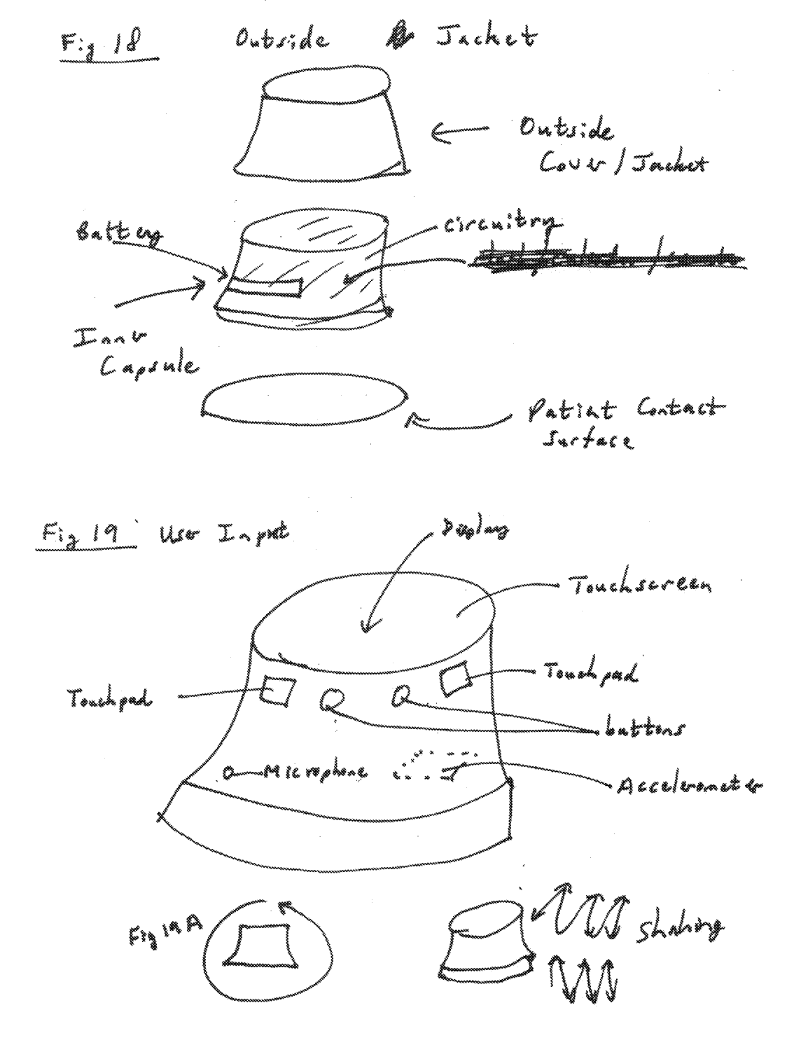

FIG. 14 shows a further elaboration on attaching the stethoscope to a stethoscope style headphone or to regular headphones. The stethoscope has a jack which allows headphone connections to be made very conveniently. Shown in FIG. 14, is the ability to connect multiple types of headphones to a stethoscope, including but not limited to stethoscope style headphones, in-the-ear headphones or earphones/earbuds, or on-the-ear or over-the-ear headphones. Regardless of the style of headphone being used, a retainer means is disclosed which facilitates the robust connection of the plug to the stethoscope, such that the plug does not easily detach from the stethoscope during normal activity. This provides an attachment force between a plug and the stethoscope that exceeds the force of attachments normally associated with a plug inserted into the connection jack. The invention includes a number of different retention means. A screw may be used to apply a force to the plug or to a conductor on the plug in order to retain the plug insertion. Alternatively, a threaded hole may be provided at the site of the female jack with a corresponding thread on the male plug in order to provide a screw attachment between the plug-in the stethoscope. Another alternative, is to provide bayonet slots in which case the plug has bayonet edges such that the plug is inserted and then rotated in order to lock the bayonet attachment. Yet another alternative is to provide locking holes adjacent to the female plug hole, with corresponding locking arms which engage with the locking holes.

The locking mechanism or retainer means, is particularly useful with a stethoscope style headphone with the combination of stethoscope and headphones worn around the neck of the user such that activity on the part of the user can result in the stethoscope becoming detached from the cable or tubing which attaches the stethoscope to the stethoscope-style headphones. As shown, a cable with plug on the end is attached to the stethoscope using one of the retainer means described previously, or simply plugged in directly, relying on the natural forces within the female jack to engage the male black firmly. At the other end of the cable or tubing, a similar or identical retainer means could be placed in a junction such that the cable or tubing can be firmly retained at both the upper end where a headset is located and the lower end where a stethoscope is located. The retention means being placed at the junction is optional and the cable tubing could be permanently attached to the junction. The benefit of providing a disengagement mechanism at both ends of the cable tubing is that if the cable tubing is worn or requires replacement, it is very simple to disengage at both ends and provide the user with a replacement part/tube without the necessity to replace or service the headset at the top end or the stethoscope at the bottom end.

The stethoscope style headphone comprises stiff tubing arms with small loudspeakers located at the top along with eartips. The stethoscope style headphones is therefore a complete electrical headphone with output transducers at the tips. The junction box can optionally include electronic circuitry or other connections to facilitate further connection or communication to other devices externally. A wireless option would provide wireless communication such as Bluetooth or Wi-Fi between the stethoscope and/or headphones and a remote device such as a mobile phone, tablet or computer or a Wi-Fi connection or

Internet connection to remote devices. A jack could optionally be included in the junction to provide a wired connection to any of the aforementioned devices and systems. The junction could further optionally include a means to connect a charger which can then charge a battery inside of the stethoscope or the junction could include primary or rechargeable batteries inside the junction to power the stethoscope or provide backup power or recharging power to the stethoscope. The junction could also include a microphone such that the stethoscope style headphone with stiff upper arm tubing styling could be used as a headset for a mobile phone. The junction could include many further functions such as voice or data recording whereby the circuitry in the Junction communicates with the stethoscope.

FIG. 15 shows a wireless link arrangements whereby the stethoscope can wirelessly communicate with wireless headphones or earphones and/or communicate with a mobile device such as a cell phone/tablet or computer. In this arrangement, a wireless stethoscope which is handheld is possible. A further wireless link or an alternative wireless link could be provided to the Internet via either a Wi-Fi or longer distance communication protocol, to facilitates tele-medical or distance listening. In the case of the wireless links connecting more than one device, a multi-link protocol is required such that multiple point-to-point wireless communication is possible.

FIG. 15 a shows a physical arrangement wherein the stethoscope or a stethoscope sensor for sensing body sounds is physically attached or coupled to a mobile device forming one contiguous unit. In this case, the stethoscope optionally includes other peripheral functions for physiological measurement as previously described.

FIG. 16 shows an implementation of a physical structure for the stethoscope wherein distinct modules can be stacked and attached to form a modular stack. In FIG. 16, 3 modules are shown, module A, module B, module C. By stacking the modules and attaching the different modules to each other they may be interconnected via connections which engage when modules are attached. The system provides flexibility in that modules may be included or removed from the stack such that module A could be connected directly to module C and omit module B, for example. The interconnections between the modules can be implemented by conventional board to board connectors or spring loaded contacts between one circuit and the circuit above or below. The module stack is secured into a contiguous device by a locking means or attachment means that holds the various modules together. Such a locking means can include, but is not limited to, screws through the modules, latches between modules, magnetic attachment, threads, or locking latches.

The modules need not be the same shape as one another and as shown module D could connect to module C even though they are not the same shape. It is further shown that module E could connect to module D. the invention therefore includes a flexible arrangement of interconnected and stacking modules.

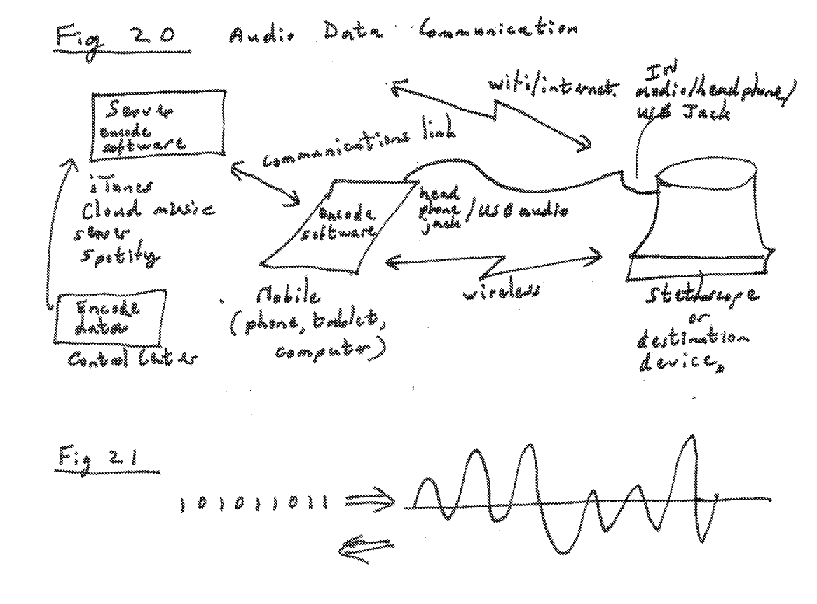

FIG. 17 shows another embodiment of the expansion capability, wherein an external module is plugged into the stethoscope. The attachment means could be a quick connect and disconnect arrangement or it could be one of the previous retention retainer means shown in FIG. 14 for headphone cables. When attached, the external modules are mechanically and electrically connected to the stethoscope body via the interconnections previously disclosed.

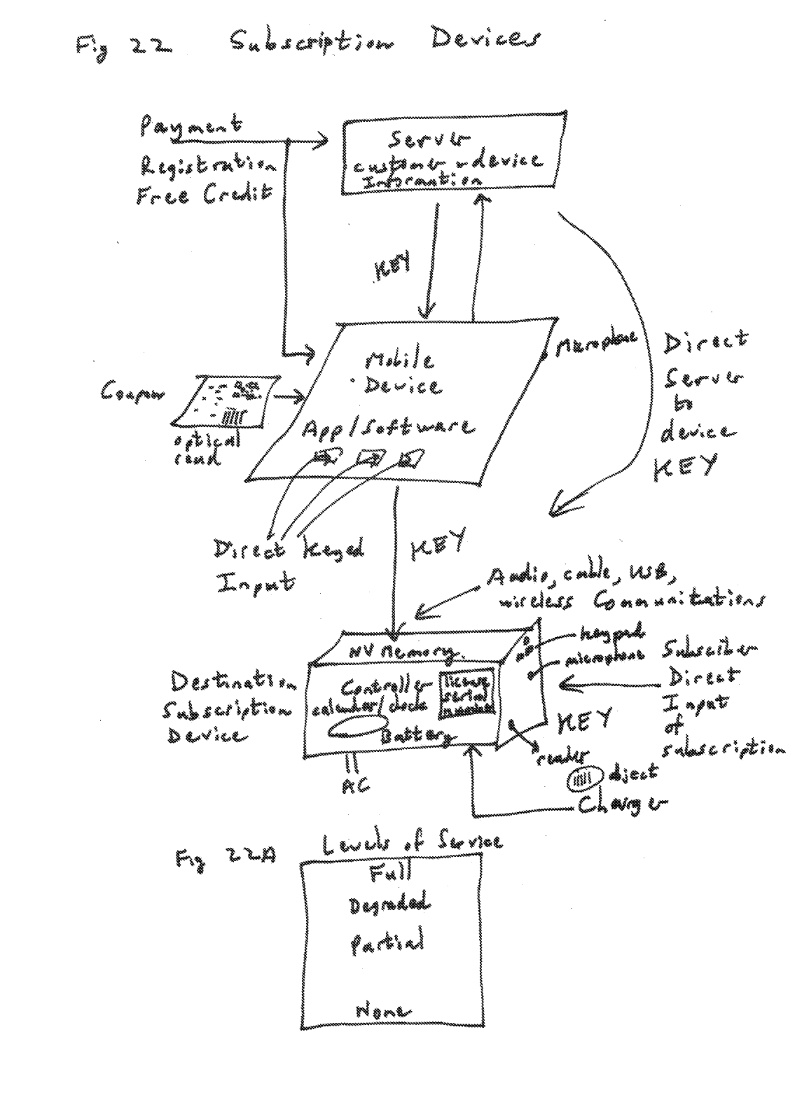

FIG. 18 shows a further elaboration of the invention of an outside protective jacket for the stethoscope. The capsule houses all or many the electronic components required for operation of the stethoscope. The capsule can be recycled in order to reuse the most expensive elements of the stethoscope. The outside cover can be replaced and covers the inner capsule. On the bottom a patient contact surface can also be replaced. The outside cover or jackets and/or the patient contact surface can therefore provide a disposable item which can then be provided in a sterile form for use in the clinical setting in order to prevent the transmission of bacteria between patients. The inner capsule can optionally include a battery, circuitry, and internal software in order to limit the usable life of the electronic stethoscope between replacements of the outer components. The electronics can optionally include a sensor that detects the attachment and detachment of the outer covers, so that the device can intelligently monitor uses, sterile change events, duration of use between changes in sterile covers, battery levels, number of uses and other usage data. Such data can then be used to monitor a report on compliance with best practices and required protocols for maintaining cleanliness. This can be further enhanced by including accelerometer data. In this method, the capsule can detect that movement of the stethoscope. It can therefore detect that the stethoscope has been moved beyond a given distance, suggesting that it is being used on more than one patient, or is being moved from room to room in a hospital. This provides a method for ensuring that a stethoscope is used only within one space or room, and/or it is being used only in one location unless the sterile cover is being changed prior to use. The operation of the stethoscope can be disabled or a warning issued to the user, to remind the user that the stethoscope has been moved, or other event has occurred, which requires the sterile cover to be replaced, or the unit to be placed into a recycling bin for re-cleaning and resetting before being used again. Such a method can be combined with the subscription methods described in this disclosure.

FIG. 19 shows a variety of user interaction methods envisioned by the present invention. Conveniently located around the sides of the housing, are pushbuttons or touch pets to elicit actions and provide input to the stethoscope. These buttons, while preferably located around the side, can optionally be placed at any convenient location on the stethoscope housing. A display is provided, which can optionally include a touchscreen interface. Internal to the stethoscope, an accelerometer provides sensing for angle, movement and orientation, which can also be used for user input. For example, flipping the stethoscope over in a rotational manner can be used to change the filtering operation such a rotational movements would be such that it would mimic the action performed when the head of a mechanical stethoscope is rotated in order to change between Bell and diaphragm mode. Similarly, the stethoscope filter sitting could be changed by such a rotational movement, as an example. Another accelerometer-based interaction could be shaking the stethoscope in order to either wake up or put the stethoscope to sleep or into the standby mode. A microphone can be included to provide voice interaction or voice directed operation or could be used for voice identification in order to identify the user.

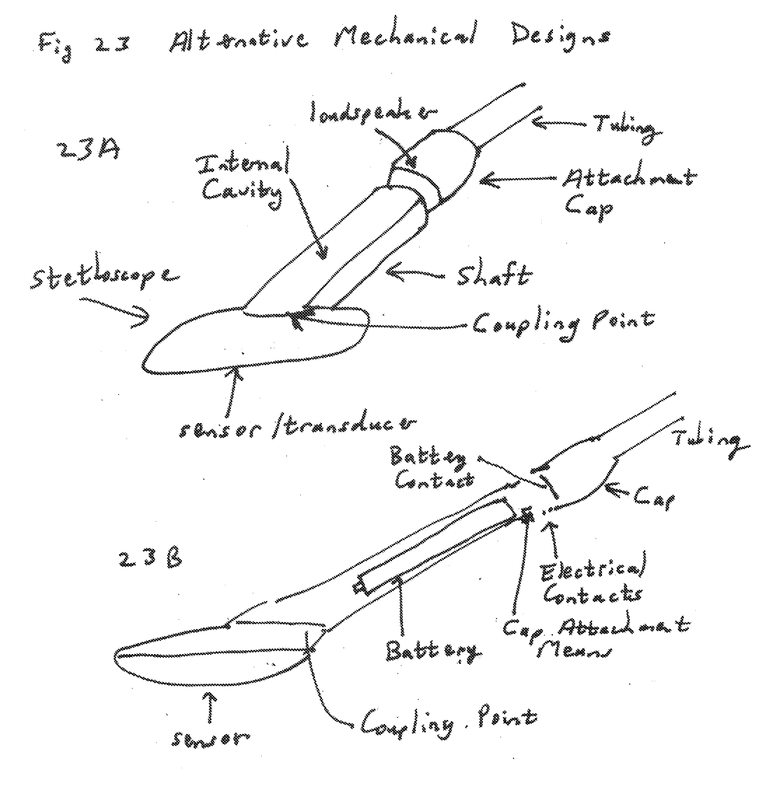

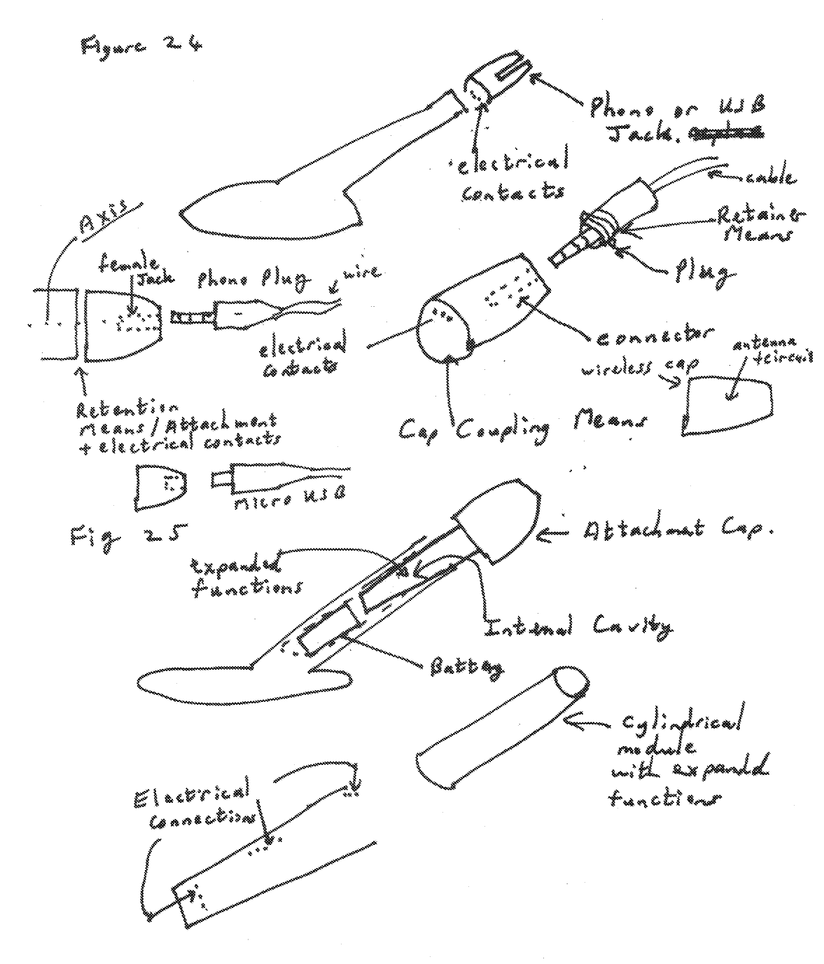

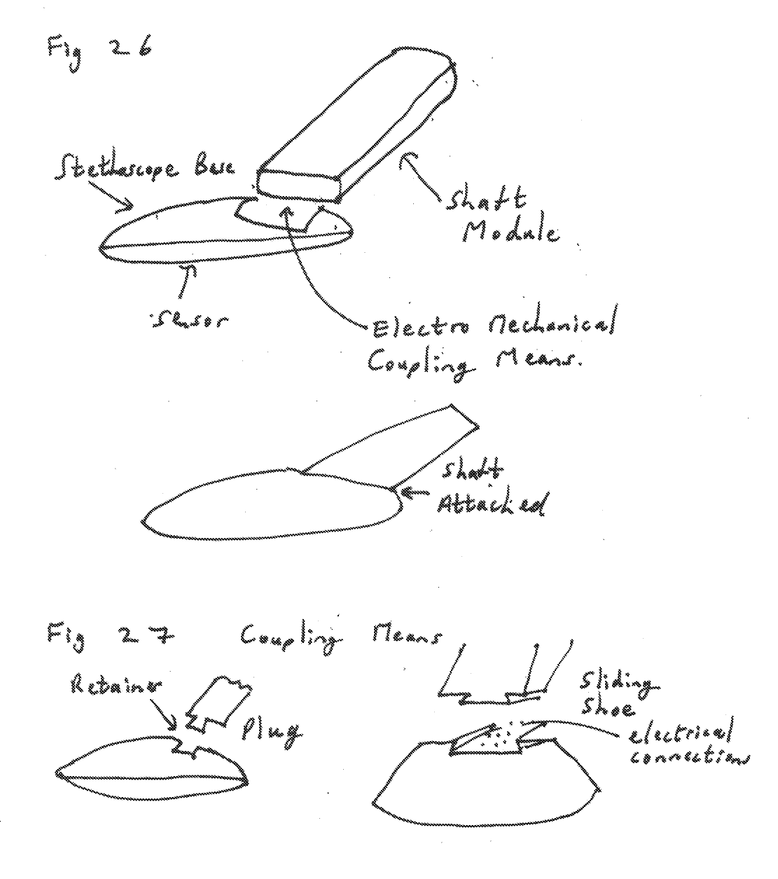

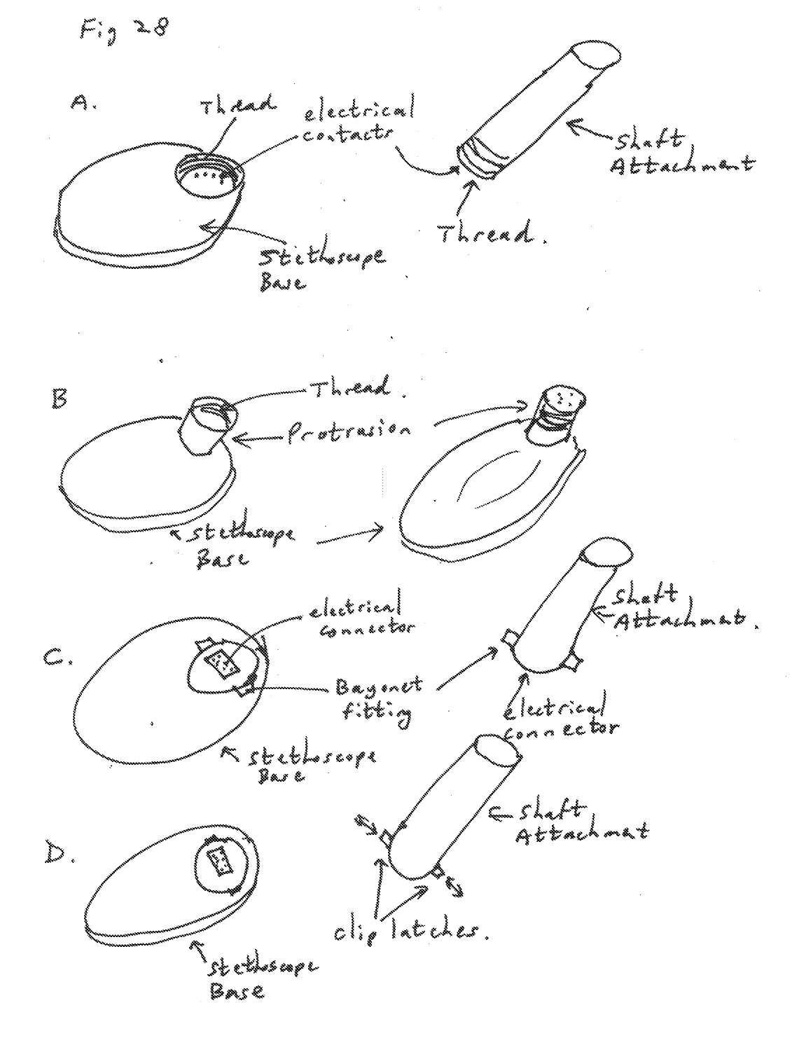

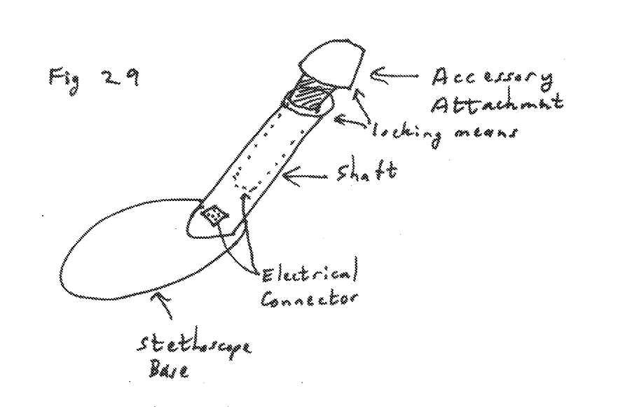

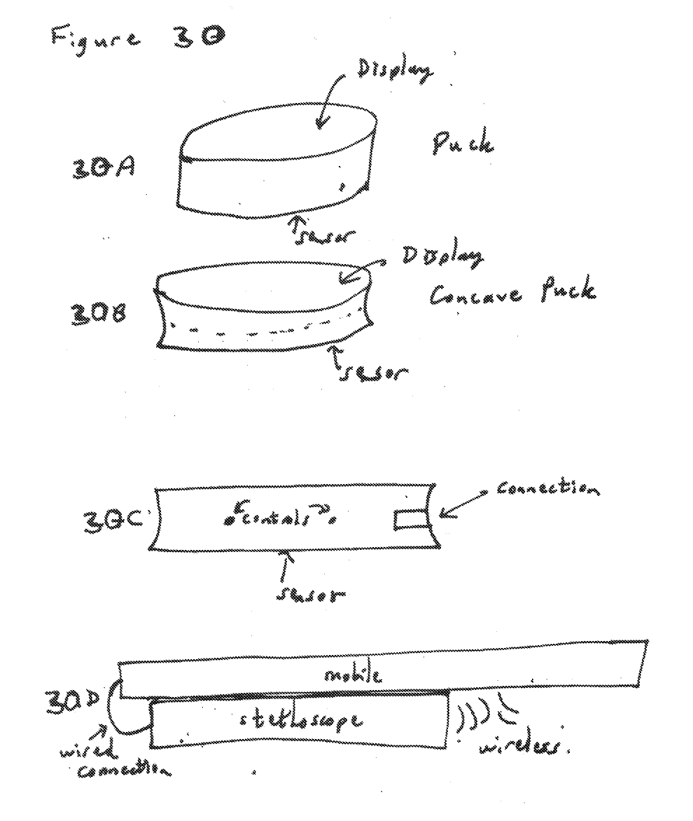

FIG. 20 further elaborates on data communications between the present invention and other equipment using audio signals as a means of data communication. The use of audio signals is extremely convenient for the reason that so many devices utilize audio and have the capability to output audio signals. These include cell phones, tablets, and computers, all of which are shown as mobile in FIG. 20. It is therefore convenient to convert digital data into an audio signal as shown in FIG. 21 in order the to be transmitted into the present invention. While the present invention is preferably a stethoscope, as shown in FIG. 20, the invention is not limited to the stethoscope and this method as herein disclosed applies to any device which has been adapted to utilize audio signals for the communication of digital data.

The steps involved in using audio data as a means of communication are shown in FIG. 20. In one method, information from a server is encoded into a digital audio signal and transferred to an intermediate or local device, labeled “mobile”, which can include a cell phone, tablets, or computer. The digital audio data file is being transferred digitally via USB audio or a wireless means such as Bluetooth to the destination device, such communication being in digital form. Alternatively, the digital audio data is converted to an analog audio stream and output on the headphone jack of the mobile device which is connected via a cable to the audio jack on the destination device. Therefore, the server has been able to send digital information in the form of a digital audio file to the destination device in either digital or analog form or a combination thereof. The server can optionally communicates directly with the destination device via the Internet, Wi-Fi, or other direct connection, circumventing the need for an intermediate mobile device.

The elegance of this arrangement is counterintuitive, thereby representing the true novelty of this communications technique. The reason is that there are a myriad of digital audio delivery services available on the Internet. Examples include Apple iTunes, Spotify, Soundcloud and other services. The ability to playback digital audio sound is also included in Internet browsing software. Therefore, the reproduction of digital audio is well supported by most devices connected to the Internet today without the need for any additional decryption software or drivers or plug-ins. The conversion of digital data into a digital audio file can be done at various sites. As shown in FIG. 20, the data could be encoded into a digital audio file or “song”, and uploaded to a server. Alternatively, a digital file could be encoded as needed on a server and the song then transmitted to the destination device either directly or via mobile. Finally, digital data could be downloaded from a server or be built into the software inside a mobile device and the data then encoded into a song or digital audio stream on the mobile device and then transmitted to the destination device by playing the song into the stethoscope when connected. The elegance of this audio encoding, is that the aforementioned music services provide for purchasing of music and songs. Therefore the user of the destination device can very conveniently purchase digital information from such widely used music servers and conveniently downloaded into his or her destination device. A supplier of digital data could therefore utilize the services of a music service which is widely used by millions of users, as a distribution medium for either free or paid digital information without any need whatsoever to invest in a specialized electronic commerce platform or acquired the end-user to install customized software in a mobile device. The song distribution method therefore provides a platform for the sale of software, features, licences, subscriptions and the like, utilizing the payment and memberships that customers establish or have already established with existing music distribution organizations. Songs could also be played to the device and detected by a built-in microphone in the device. In such a method, digital content, subscriptions or information could be transferred to a device in live events, over the phone, or at a specific location, without the need for other forms of wireless communications and complex protocols.

FIG. 21 shows a schematic form of encrypting and decrypting between digital form an analog form. The challenge of incrementing digital information into an analog audio stream is that the average or DC components of the analog stream can be offset and make it difficult to decrypt the analog audio information the invention provides for the ability of the audio encryption software to encrypt the digital data or convert the digital data into an analog audio stream such that the final analog audio stream can be decrypted with very low cost software or hardware techniques as one example method, the resulting audio stream can be compensated such that 0 crossings may be used conveniently to decrypt the analog stream into the digital data stream by creating encoding that compensates for baseline or 0 DC drift.

A further form of audio encryption is performed by embedding message data within a sound track of a video or the sound in a song. In such a case, digital message or control data is hidden or encoded into the regular sound stream and may be inaudible or audible to the listener. In such encoding, the receiving device parses the incoming audio stream and filters and decodes the audio in order to eliminate the music or soundtrack, in order to retrieve the digital message. The message than then be used to implement control or messaging functions in the receiving device. Such a method is suited to sending control messages to a device in synchronization with a video or song. This can be implemented over the internet, or can be done in a live setting, in which case a receiving device could receive the message via a microphone and react in real time to special encoded sounds that are being sent out on the public address or auditorium sound system. Such messages could include licenses or subscription keys and messages, coupons, or other messages that carry with them rights to obtain or purchase an item of value. This allows an advertiser or organization to send out rewards or offers to a group or individual based on their presence at a given time and place or event. The same method can be applied to radio or television broadcasts, or over the internet. The method allows for sending digital data or messages via an audio stream for any purposes whatsoever.

FIG. 22 shows another aspect of the present invention, which is to provide subscription devices that are enabled or disabled from performing certain functions based on a subscription being active or inactive. In FIG. 22, the subscription device has been labeled destination subscription device, and may be an electronic stethoscope, other medical device, a recreational device, a kitchen appliance, or any other electronically controlled device that performs functions or contains multiple features.

FIG. 22A lists the levels of service which could be operating at any given time in a subscription device. At the top level is full operation wherein all features are functional. At the bottom level, no features whatsoever are operational and the device does not perform any functions. At intermediate levels, certain features could be partially enabled or provided at a degraded level. FIG. 22A is not intended to be a comprehensive list of levels of service but exemplifies the fact that there are different levels of subscription that could be embodied and provided within a subscription device, subject to the status or level of a subscription service at any given time.

In FIG. 22 the destination subscription devices is shown. The device may include an internal battery or maybe powered by external AC power. There is an internal controller which controls the functions of the device and is also a control mechanism for controlling the level of service available to the user. If the device is battery operated it may include a rechargeable battery and part of the subscription control may include the ability to recharge the battery or not. Internal to the device exists license data, serial numbers, or other data structure, which allows the subscription device to be identified and to store levels of service, permissions, subscriptions, or any other relevant data to determine the level of service at any given time.

The present invention includes various novel methods for enabling and controlling the subscription service offered by the subscription device. Fundamentally, the subscription device has its functionality controlled with the reception of a key. The key sets the level of service, the duration of service, the number of times a service can be performed, or other aspects of the extent to which the subscription device may render functions or services.

At the simplest level, a subscriber enters information directly into the device using a keypad or other input means and this provides the key to the device. The input means could be mechanically operating a keypad, transmitting a sound to the device which is detected by a microphone located to the subscription device, a wireless technique such as NFC, or optical techniques such as reading an image. The subscription device may include a calendar or clock implemented in hardware or software or a combination thereof, such that the device can monitor and track time-based subscriptions and features. The license, serial number, clock and calendar data is stored in non-volatile memory so that even under conditions of loss of power, significance subscription-relevant information is not lost. In the events that power loss results in the inability to track calendar or time information, the invention includes decision-making capability by the software built into the subscription device in order to determine whether the subscription is disabled and synchronization with a reference clock on the Internet or mobile device is required in order to reactivate the subscription device, or a short-term or emergency allowance is made to allow the device to continue in a full feature or partial feature mode until the calendar time and subscription information can be re-established.

At the next higher level of the subscription system, the user may interact with the subscription device using a mobile device such as a cell phone, tablets, or computer. The mobile device communicates with the subscription device via cables or wireless, using an audio data communications method as previously described, other analog signaling or digital signaling such as USB. Wireless communication is also possible using such mechanisms as Wi-Fi, Bluetooth, or NFC communications, to name a few common protocols. Using a mobile device, the user can enter subscription information via an app or software, using direct touch-screen input, or using other input means available on the mobile device, such as capturing an image or a sound or using biometric identification such as fingerprints. Such images could include labels, coupons or photographs. Audio information could be any sound such as music, advertising, voice, a live event, or any source of sound. The software or the app in the mobile device then provides the key to the subscription device.

At the next level, the subscription information could emanate from a server. Information could be sent to the server to control the subscription information of the destination subscription device and the server could then send the subscription key directly to the destination subscription device via the Internet or it could send the key to the mobile device, which would then send the key to the destination subscription device. The server level information required to trigger a key issuance could be payment information sent to the server to inform the server that a subscription has been paid, some form of registration information could be sent to the server, or some information regarding a free credit could be sent to the server, triggering the server to send a key to the destination subscription device either directly or via an intermediate device.

A further level of complexity could be provided wherein information is entered into the mobile device as previously described, that information is uploaded to a server, which verifies the information, and then sends a key either directly or via the mobile ultimately to the subscription device.

The enabling of the device requires an interaction of multiple pieces of information. Within the device, there may be serial number or key decryption software to match key and serial number in order to ensure that a key has been targeted at a given device and not generally. A database or data structure of license numbers can be stored in the device at the time of manufacture or distribution or sale, or other stored information some of which is accessible to the user and some which is not. The keys that are sent to subscription devices could be general in nature, or they could be encrypted such that the key is only applicable to a given device based on the serial number or the licenses that are stored in the device.

When the device is unlocked and the level of service is established based on the subscription level, operations can be limited by time or date, the number of times a given operation is permitted to be performed by the device, or a complex combination of time, date and level of service can be encoded and enabled within the given key.

Alternatively, the destination subscription device could have a built-in reader or communications device which allows it to autonomously determine whether to perform a function or not. The decision or key could be determined by a separate object being used or coming into proximity with that subscription device. For example, a device could operate or not operate depending on whether an object with a specific barcode was inserted into the device or not. For example, in the case of a medical device, some consumable object could be detected, attached or inserted into the subscription device and the device would know that a valid or approved consumable had been purchased and was being used and the subscription would be unlocked only on condition that the approved consumable was being used. Similarly, in a kitchen appliance, a food ingredient or product with a specifically required label would be detected by the subscription device which would then enable the device to operate. The appliance manufacturer could therefore ensure that the appliance is only used or available for use when the customer purchases or obtains specific ingredients.

There are multiple methods envisioned by the present invention for unlocking the subscription device and enabling various functions. When used with a mobile device, a code could be manually entered into the keypad or touch screen of a mobile device and the key could be transmitted to the subscription device just as it would be if it were keyed into the keypad of a subscription device such equipped. In the case of an optical input, the mobile device might read a label or barcode, an optical code, a photograph of a specific location, a photograph of a specific object or location, or said picture being taken within a given time window. A GPS key could be created by the mobile device being present in a certain place in a certain time range or time/date window of time. Subscriptions could therefore be enabled based on what somebody photographs and/or where they do it and/or when they do it. Likewise, audio inputs could be used based on merely the audio content or the audio contents being captured in a certain place or within a certain window of time. This means of enabling a subscription device or service allows organizations to reward users based on incentives with a request to attend a certain event, visit a certain location, purchase a certain product, all performed irrespective of time or within a requested time window.

The ability to generate a key for a subscription device based on all of these inputs could be built into an app or software within the mobile device, or the information could be sent in direct or encrypted formats to a server, the server could verify the information, the validity of the request, correspond the information to the identity of the customer or the subscription device, the subscription devices serial number, license data, or other information, and generate a key accordingly.

The subscription device method disclosed in the present invention has wide applications for novel business models and device control in which the production cost or selling price of a device or service can initially be subsidized in return for future activities by the customer or purchasers of associated goods and services.

Another method for establishing a subscription includes the steps of a user establishing an account on the server, registering the subscription device in connection with the specific user's account, and providing payment information associated with the account and the device. Such payment information could take the form of linking a credit card or bank accounts as a form of payment from which the server could withdrawal monthly, quarterly or annual subscription amounts. Alternatively the user could submit other information in order to redeem free credits in lieu of monetary payments for the use of the device. As a further alternative, a third-party could submit credits or payments on behalf of the user. Any and all such combinations could be used over time to continue to provide services.

payment method is included in the present invention wherein advertising promotions or educational credits can be applied towards payment of a subscription. In the advertising method, a user agrees to view advertising content, possibly in an interactive manner in order to validate to the advertiser that the user has read or watched or listened to the advertising content, understood the advertising content, after which the advertiser automatically submits or manually submits payment information to the server in order to issue a key to enable the features and functions performed by the subscription device. In the educational method, educational content is presented to the user, the user participates in an interactive question and answer test, and upon passing the test, credits are applied to the users accounts in order to be used for continued subscription, triggering the issuance of a key. In some cases, methods of payment may be combined such that a user might pay for an ongoing subscription in combination with free credits being applied with advertising or educational programs or a combination of all three payment forms. The invention includes all of the steps defined above, or a subset or other combination of such steps in order to trigger the issuance of subscription keys.

The subscription device may take the form of a physical device, but it can also take the form of software with various features and functions. For example, such feature and function unlocking can be applied to apps on mobile platform devices. In this case, any of the methods disclosed herein result in the transmission or decoding of a license key, with then enables the software App to function at various levels.

the specific case where the subscription device is an electronic stethoscope, some or all of the above subscription methods can be applied to the enabling or disabling of functions of the stethoscope. Specifically, a subscription could be applied wherein the allowable volume levels of the stethoscope audio outputs can be controlled by a subscription. When the subscription is valid, the full volume range could be enabled. At a lower level of subscription, the volume level could be limited, or if a lower-cost plan subscription were in place, a limited range of volume could be implemented and enabled by the software built into the stethoscope. Stethoscopes might be provided to a user with a higher level of functionality and as time passes, or after a limited number of uses, the volume level could be degraded to that of a conventional stethoscope, making it equivalent to a non-electronic stethoscope, or setting the volume to a level somewhat higher than a mechanical stethoscope, but not sufficiently high to obtain the full benefit of the device at full performance.

Since the stethoscope is a medical device, it is undesirable to completely turn off a function. The present invention allows for a feature such as volume level to be slowly degraded over time, allowing the user to be warned that the subscription period is about to end or is nearing completion, and giving the user the opportunity to reestablish service prior to complete loss or degradation of the function. Optionally, a given feature could be degraded to a certain level either for a limited amount of time or permanently. In the case of volume control, the level of output could be limited to a low level once the subscription has expired.

Some other functionality could be applied to filtering the audio signal. At a full subscription level, a multitude of filters could be enabled in the device or downloaded into the device, whereas at a low limited subscription level the range of choices or the filtering capability could be limited or curtailed. Again, the invention provides for the opportunity to limit or degrade the functionality over a period of time in order to warn the user that the subscription is about to expire or has expired and to provide the user with the opportunity to re-establish service before all functionality is lost.

Another way to degrade the operation of the stethoscope without complete loss of use is to limit the duration of time that the stethoscope is turned on under circumstances that the subscription is about to end or has ended. For example if the stethoscope is normally used for a minute or more, when the subscription is ending or has ended, the on-time of the stethoscope could be limited to 20 seconds, 30 seconds, or 40 seconds.

further method for restricting use of the stethoscope by a subscription model or limited time use model, would be to control the capability to charge the internal rechargeable battery. When a subscription or service has expired, the charging circuits in the stethoscope could be completely disabled, or a limited amount of charge could be permitted in order to provide limited use of the stethoscope. A one time use subscription could be implemented by either restricting the permission to recharge an internal rechargeable battery or by enclosing a primary cell inside the stethoscope, requiring the device to be physically opened in order to exchange the battery. The benefits of a one time use or limited use stethoscope is that a reusable capsule as described previously could contain the battery and charging mechanism or a another subscription mechanism to ensure that the stethoscope is used in a limited manner and encased in a new sterile housing prior to repeated use.

Therefore, the stethoscope could be provided with a full range of volume control and a broad range of filtering capability and at such time that a subscription is about to end or has ended, either volume range could be limited, or filtering could be limited, or the amount of time that the stethoscope is allowed to operate each time could be limited, or a combination of the above.

In the case of the stethoscope, a subscription key could be transmitted to the stethoscope using encrypted audio in the form of a “song” or sound sequence, which is played from the output of a mobile device or other source into the stethoscope. The stethoscope then decodes the digital data encrypted in the audio signal to enable or disable functions and features accordingly.

In the specific case of the stethoscope, or other medical device, the sponsored advertising and educational credits payment methods described above could be provided by an advertiser wishing to reach the specific demographic group that uses the stethoscope or other medical device in question. This method of payment and sponsorship could also be applied to patients who are using a device wherein the patients could make partial payments or no payments at all, and a sponsor could apply credits according to the patients participation in advertising programs, medical studies, or educational programs. A health insurance organization could use this system and method for partially or fully paying for or subsidizing the use of the device in healthcare by patients or clinicians. In this method, the insurance organization can additionally monitor the use of the subscription device access information and measurement information from the device solicits or instruct the user to participate in advertising or educational programs upon the completion of which the subscription device is continually enabled, configured, or disabled.

database can be maintained of the user profiles, allowing organizations or advertisers to provide subscription credits or services to a targeted group. The group might be employees of an organization, or a professional group such as doctors, specific specialists, nurses, students, children, adults, and the like.

Subscription devices have, inherent in their functionality, the opportunity to be “hacked” i.e. subscription devices being enabled under fraudulent circumstances. It is therefore critical for the transmission of validation or subscription keys and the management of validation keys to be securely controlled. There are various methods for doing so. One method included in the present invention is for license keys to be stored in memory within the controller semiconductor device in the subscription device. Such license keys are programmed into the subscription device during the manufacturing process, distribution process or at the time of sale or shipment to the end-user. Corresponding subscription keys are then stored in the server computer and transmitted to the subscription device according to the subscription to be enabled. By pre-storing the license keys inside the subscription device and storing the equivalent unlock keys at the server, it would be extremely difficult for a hacker to enter the correct subscription enabling key into a subscription device, since the keys could be entirely random number sequences that cannot be derived from any algorithm. In order for this method to work the controller software within the subscription device would need a method such that the downloading of a certain number of invalid subscription keys would result in degradation or disabling of the features and functions performed by the subscription device. The lockout of so-called lucky guesses in the pre-storing of random number subscription sequences would mean that there would be no algorithmic method for computing the keys for a given device. Within the controller within the subscription device, the memory containing such license keys would either be internally encrypted or would be inaccessible from the memory.

In order to provide good customer service, in the event that the server is temporarily unavailable or some other event has resulted in the subscription device been rendered inoperable, the invention also includes a method wherein a universal unlock key is included in a lot or an entire class of devices, such that the user could enter the universal or emergency key and enable the device for a short period of time under such unusual circumstances. The emergency or universal keys could be used for a limited number of times only. Therefore, even if a user is told the universal or emergency key code, it would be of very limited use.

Another aspect of the subscription device key is the provision for converting the subscription device into a perpetual use device that no longer requires another key and is permanently enabled for perpetual use. The method therefore includes the feature that subscription devices may contain one license key or multiple license keys that are unique, in that they provide a selectable level of service on a perpetual basis. A user could therefore purchase or be given a subscription device which has a limited time use, always operated on a subscription basis, whereby ongoing subscription keys are required. Then, at some point in the future, the user can elect to pay a fee in order to perpetually own the device as a fully functioning unit at a given level of service. Alternatively, the user might use the device on a subscription basis for a limited amount of time at the end of which a perpetual use key permanently unlocks the device at a particular level of service or set of features and functions.

Instead of using a calendar, clock or “use counter” in the device, the use of a battery can be used as a low cost proxy for the amount of use or the amount of time that the subscription devices used. The subscription device can then simply allow the battery to discharge and the number of re-charges that are permitted can then be used as a control mechanism for limiting the use of the device. This can be achieved by the internal controller limiting the amount that the charger may be used to recharge the battery, or the controller can monitor the battery voltage and ensure that the battery voltage is allowed to decrease, but detects recharge at any time that the battery voltage increases. This can be useful in the case of a device that uses either a replaceable primary or rechargeable battery. The battery voltage can be monitored by the internal controller and then written to non-volatile memory. If the dead batteries are then removed and replaced, the controller can read previous battery voltages, compare them to the current battery voltage and determine that the battery voltage is increased, and therefore the battery has been replaced with a fresh battery. The controller software can then check with the permissible use rules of the subscription plan and determine how many times the battery voltage may be replenished or how many times the battery may be replaced by the user before the subscription expires. This provides for a very low cost mechanism for validating or invalidating a subscription device, and obviates the need for any communication between the subscription device and a server, mobile device or any other external system or the need for a calendar or clock internal to the device. Of course, the battery voltage or charging algorithm can be used in combination with an ongoing subscription system managed by an external mobile device or server.

The invention therefore allows for a wide variety of subscription management methods including, but not limited to, direct control and entry of keys, control of power source, transmission of a key from a mobile device or a server, and payments and redemption methods for subscriptions based on purchasing specific products associated with the subscription device or not associated with the device, participation in advertising or promotional programs, activities, medical studies or other activities or educational programs or outright payments for services.

With regard to payment services, it is extremely common for large populations to have mobile telephone accounts. Subscription devices, especially those that are used in conjunction with mobile devices, could have their subscriptions managed by the same organization that manages the subscription services for a mobile device. For example, a medical or healthcare device that is used in conjunction with a mobile device, or separately, can become part of a bundled mobile telephone account. This provides the mobile service provider with a source of revenue for multiple devices used by its customer base. Similarly, any other ongoing service providers such as utility companies or cable subscription companies or banks or credit card companies can also manage the payments and license keys for subscription devices. This method allows device companies to create subscription devices and use the distribution and existing infrastructure of subscription management organizations to manage the payment processing and subscription keys.

FIG. 23 shows an alternative mechanical design for a stethoscope. In FIG. 23A, the stethoscope comprises a sensor or transducer surface at the bottom, which comes into contact with the patient in order to detect body sounds. The stethoscope body includes a shaft, which emanates from the base of the stethoscope, usually at an angle, and is connected at a coupling points between the shaft and the stethoscope body. The shaft and the stethoscope body can be formed in a unified piece. The shaft can be hollow and provide an internal cavity, which may be occupied by a battery. An attachment is attached to the shaft using a attachment means thereby making electrical contact as shown in FIG. 23B to connect a terminal of the battery to the stethoscope electronics. The attachment cap or cap may further contain a loudspeaker and be acoustically coupled to a tubing which goes up to a headset for a listener to listen to stethoscope sounds which are captured by the sensor, amplified by the stethoscope electronics, reproduced by the loudspeaker, which then reproduces the sound which travels up the tubing to the listener's ears. The structure shown in FIG. 23 offers a number of opportunities for novel inventions to be added to the stethoscope with this mechanical design.

FIG. 24 shows one novel invention for use with the shaft stethoscope structure. In this invention, the attachment at the top of the shaft no longer contains a speaker and tubing attachment. Instead, the cap contains a phono, USB or other jack located at the end of the cap where the tubing previously existed. A plug can then be connected or plugged into the jack such that a cable can be connected to the attachment cap instead of using a tubing. Such a cap therefore eliminates the use of an acoustic tube and replaces it with an electrical connection to the stethoscope, providing for an electrical output of the stethoscope audio signal in place of an acoustic output. The invention further includes a retainer means as previously described wherein the plug can be attached to the rear cap in a robust manner to resist removal of the plug from tensile forces on the cable. The new invention of the attachment cap therefore converts an electronic stethoscope that was previously used for acoustic output of an audio signal into a stethoscope which is used exclusively for electrical output of the audio signal. It should also be noted that the electrical connections can also provide for other functions as previously described with electrical contacts between the cap and the stethoscope being made when it is attached to the stethoscope.

Note that in FIG. 24, it is shown that the phono plug or USB plug and jacks are coaxial i.e. along the same axis, as the shaft. Some angular deviation is permitted, however the connection is placed such that the hollow tubing of a conventional stethoscope air-coupled headset is clearly omitted and replaced by electrical connections. This is a critical step to converting the more conventional air-coupled stethoscope into a handheld, more compact structure, while maintaining other aspects of this physical design. The modification allows even previously manufactured stethoscopes that use a loudspeaker and hollow tubing air conduction of sound to have the shown attachment act as a field replacement, wherein the hollow tubing is replaced by generally available headphones or a direct USB connection, and the tubing attachment dispensed with, while maintaining full use of the remainder of the stethoscope. The novelty of the invention so described includes the economical and elegant change that is possible such that even a previously manufactured device can be adapted for use with electronic output means. The same can be applied to adding a wireless communications means housed within an end cap, such that the tubing is removed, the new cap is attached, and hollow tubes are replaced by a wireless communications device, again making a large cumbersome stethoscope into a compact handheld device.

Another novel invention is shown in FIG. 25, wherein the internal cavity in the shaft can be used to house functions beyond merely a battery. A smaller battery can be inserted into the shaft in combination with expanded functions, which occupy a volume along with the battery. In this case, the internal cavity provides volumetric space for both functional electronics and a power source. The power source and the expanded functions can be contained in a cylindrical module which is inserted into the cavity, or they may be attached to the attachment cap. Such functions include physiological measurements, communications, storage, or other functions previously discussed in association with expansion modules that may be attached to a stethoscope. The internal cavity of this stethoscope structure therefore provides a space into which expanded functions may be added, additional electronics or power may be contained. The electrical connections to these expanded functions could be provided within the attachment cap or they can be included within the cavity inside the shaft.

FIG. 26 shows yet another novel expansion to this basic stethoscope structure. In this configuration, the stethoscope base includes the sensor and core electronics of the stethoscope and a shaft module, which is a separate element, is attached to the stethoscope base to form a contiguous structure. The shaft module is attached to the stethoscope base via an electromechanical coupling means. The coupling means provides both a mechanical attachment and electrical connection between the shaft module and the stethoscope base.

FIG. 27 further expands on the coupling means the coupling could be via a plug wherein the plug provides electrical connection and a retainer means is included to prevent the shaft from disengaging from the stethoscope base. Another method is to provide a sliding mechanism, wherein the shaft module slides into mating grooves such that the slide and cruise coupling provides mechanical retention and stability and electrical contacts are placed at the base or at the end of the groove. There are other methods for providing an electromechanical coupling means and the invention includes all such attachments, the primary inventive step being that the shaft of this stethoscope structure can be used for more advanced functions then merely containing a simple primary cell.

It should be noted that the shaft may be significantly shortened and reduced in volume compared to the shafts typical of electronic stethoscopes of this structure. For example, if the shaft contains an electrical connection such as a phono or USB jack, potentially even excluding any source of power, this would require an extremely small and compact shaft, significantly smaller than is customary with this stethoscope design. In such cases where the source of power is not in the shaft, it may be provided with a rechargeable battery located inside the stethoscope base, or the stethoscope could be powered via the electrical connection from an external source. Such a modification to the stethoscope wherein an external source of power is provided and either a very small or no internal power source is included in the stethoscope may be of interest in situations where the stethoscope is used in conjunction with an external device such as a mobile device or computer or tablets which can provide electrical power to the stethoscope for the purposes of sensing body sounds.

FIG. 28A shows further details of the attachments of a shaft attachment to the stethoscope base in an alternative embodiment of the present invention. The shaft attachment contains an extension accessory or an expansion connection means for expanding the functionality or connectivity of the stethoscope base. Such functional extensions or expanded connections have been described earlier, such as but not limited to headphones, USB digital audio, wireless communications functions, external storage, physiological measurements, and other expanded functions. In the embodiment shown, the shaft and the stethoscope base are both threaded such that the shaft attachment can be screwed onto the stethoscope base upon screwing the shaft attachment into the stethoscope base or on to the stethoscope base electrical contacts make electrical connection between the shaft attachment and a stethoscope base. FIG. 28B shows that this arrangement includes variations on this attachment wherein there can be some extension or protrusion above the stethoscope base, and the threads can be internal or external. In FIG. 28C, a further alternative embodiment is shown in which the shaft attachment is attached to the stethoscope base using a bayonet fitting, wherein the shaft attachment clips into place and an electrical connection is made between connectors located on the stethoscope base and on the shaft attachment, said connectors being mating pairs. In FIG. 28D, the shaft attachment clips into the stethoscope base using a latching mechanism. Note that the present invention includes any attachment means between a shaft attachment function or module and a stethoscope base, and FIG. 28 is merely illustrative of a few alternatives.

In FIG. 29, the shaft is fixed to the stethoscope base and the accessory attachment is inserted into the volume of the internal cavity of the shaft. At the base of the internal cavity of the shaft is an electrical connector and the accessory attachments has a mating electrical connector which makes a connection between the accessory attachment and the stethoscope base when the accessory attachment is fully inserted into the shaft. When the accessory attachment has been fully inserted into the shaft the accessory attachment and the shaft have a matching locking means for insuring that the accessory attachment remains in place attached to the shaft and stethoscope base. The locking means may include a thread for making a screwing attachments, a latch that can be easily released, a bayonet attachment, or any other means of firmly positioning the accessory attachment. The accessory attachment may include a power source such as a rechargeable battery, in which case the accessory attachments can be removed, placed into a battery charger, while a 2nd battery accessory attachment is inserted into the shaft to use the stethoscope. This provides a convenient method for charging spare batteries without loss of use of the stethoscope. Alternatively the accessory attachment could include a power source as well as other functions, as previously disclosed. One of the significant benefits of the accessory attachment, is that it allows an embodiment as shown in FIG. 29 to form a handheld stethoscope that explicitly excludes the use of conventional stethoscope tubing as the means of listening to the stethoscope sounds. The novelty of this embodiment is that other means of listening can be done such as wireless audio communication and/or headphone outputs listening, while eliminating the cumbersome tubing which is normally required for air-conducted audio transmission to the listener's ears.

It should be emphasized that the simplest novel invention associated with the stethoscope base and shaft structure is the replacement of the closing cap and tubing attachment with built-in loudspeaker, with a simple jack at the end, in place of the tubing, such that headphones can be connected to the stethoscope. Such a cap eliminates the tubing connection, resulting in a very compact device that no longer requires a tubing-based stethoscope-style headphone as is used on mechanical stethoscopes.

FIG. 30 shows a further mechanical structure for building a stethoscope. The structure offers the advantage of a very elementary shape which can provide for a very compact design. In FIG. 30A, a puck design is shown wherein the stethoscope is merely a cylindrical device in a cylindrical housing with a sensor on the bottom surface and a display optionally placed on the upper surface. In FIG. 30B, the puck is modified in its cylindrical shape to provide a concave profile to the cylindrical shape in order to facilitates conveniently holding the stethoscope. In FIG. 30C, the location of optional connection and controls is shown around the circumference of the cylinder. In FIG. 30D, the cylindrical stethoscope is shown coupled to a mobile phone illustrating a benefit of the cylindrical design wherein it can be easily attached to a mobile phone and the combination being a narrow profiled solution. The connection between the mobile phone and a stethoscope can be achieved by an optional wired connection or wireless communication means.

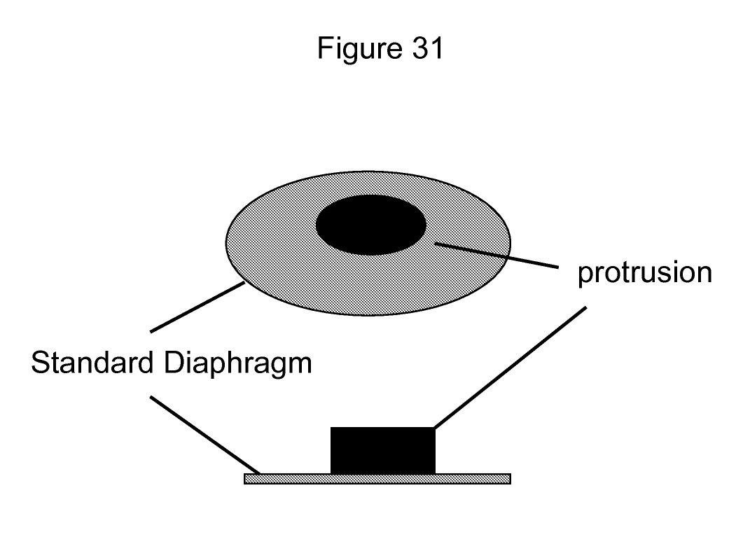

The present invention can be further enhanced by the attachment of a neonatal stethoscope diaphragm attachment as shown in FIG. 31. The neonatal diaphragm comprises a flat surface the same or approximately the same size as the adult or standard diaphragm used on the stethoscope. However, placed on the flat surface of the diaphragm, on the surface that is typically placed in contact with a patient or other vibrating body, an additional protrusion element is attached. This protrusion element protrudes from the flat surface between 1 mm and 15 mm above the flat surface, so that one surface of the protrusion can be placed against a vibrating surface so that vibrations are coupled from the vibrating body, via the protrusion, to the diaphragm.

The protrusion has a smaller contact surface area than the standard diaphragm, allowing the surface area of the vibration coupling between vibration body and diaphragm to be effectively smaller that it would be with the standard diaphragm. This is an important feature in the sensing of body sounds from infants and small children, or in sensing sound from surfaces to which coupling via the standard diaphragm is sub-optimal. This could also include adults who have uneven body surfaces, such as ribs, or for sensing sounds from flat surfaces such as walls or curved surfaces such as pipes, tubes, or other uneven surfaces. The diaphragm with protrusion that improves the versatility and sensing capability of the present invention so that vibrations from small, flat or uneven sensing surfaces can be detected.

The present invention includes numerous structures, methods and novelties applicable to stethoscopes, medical devices, and for some methods, to any electronic device. While the descriptions provides specific details of specific methods applied under specific conditions, the invention covers any combination of structures and methods disclosed herein, as well as combination of the above methods with use in conventionally structured stethoscopes wherein the traditional tubing or wiring is connected as in FIG. 1 or other variations on the specific physical design of the stethoscope or device.

1. An electronic stethoscope comprising:
transducer positioned to make contact with a body to convert the body's mechanical vibrations to electrical signals;
one or more internal electronic sub-assemblies;
detachable wired or wireless connection means to facilitate communications between internal electronics and external devices;
external devices comprising at least one of wired headphones, wireless headphones, hearing aid, mobile device, computer, wireless receiver, communications network;
wherein transducer, electronic sub-assemblies, and connection means are internally connected, mechanically stacked and housed in a single housing.

2. An electronic stethoscope as claimed in claim 1 further including a rechargeable battery internal to the housing with a wired or wireless means to recharge the battery from an external power source.

3. An electronic stethoscope as claimed in claim 1 further including an electronic display means.

4. An electronic stethoscope comprising:
a housing;
vibration transducer placed in the housing and positioned to sense vibrations from a body;
electronics placed inside the housing to process sounds from the sensor;
a cavity within the housing for placing a removable power source, electronics module or a combination of electronic module and power source;
a first cover attachment containing a loudspeaker and attached to a stethoscope tubing and hollow tube headset such that sound from the loudspeaker can be acoustically coupled via the tubing and headset to a listener's ears;
a second cover attachment that contains electronic communications means for transmitting signals to sound production means external to the housing;
a third cover attachment that closes the cavity to retain one or more electronics modules placed inside the cavity;
wherein a user can selectively close the cavity with either first, second or third cover attachment.

5. A stethoscope as claimed in claim 4 wherein the second cover attachment includes an electrical connector to connect external headphones to the stethoscope.

6. A stethoscope as claimed in claim 4 wherein the second cover attachment includes wireless communications means to wirelessly transmit sound signals to at least one wireless receiver.

7. A stethoscope as claimed in claim 4 wherein one or more power sources and one or more electronic modules can be placed inside the cavity.
